(19) United States
(12) Patent Application Publication
(10) Pub. No.: US 2025/0362316 A1
(43) Pub. Date: Nov. 27, 2025

(54) APPARATUS AND METHOD

(71) Applicant: THE UNIVERSITY OF LIVERPOOL, Liverpool (GB)

(72) Inventors: Simon MAHER, Liverpool (GB); Yufeng ZHOU, Liverpool (GB)

(21) Appl. No.: 19/136,510

(22) PCT Filed: Dec. 21, 2023
(86) PCT No.: PCT/GB2023/053348

§ 371 Date: Jun. 6, 2025

(30) Foreign Application Priority Data
Dec. 21, 2022 (GB) 2219434.4
Dec. 30, 2022 (GB) 2219856.8
Jan. 10, 2023 (GB) 2300354.4

(51) Int. Cl. G01N 33/94; G01N 30/95

(52) U.S. Cl. CPC G01N 33/94; G01N 30/95

(57) ABSTRACT

A method comprising:
providing a sample comprising analytes, for example in a matrix, on a substrate, for example a monolithic substrate, comprising a stationary phase;
separating the analytes, for example mutually and/or relative to the matrix, on the substrate using a mobile phase; and
analysing the separated analytes using mass spectrometry, wherein the substrate provides, at least in part, an ion source for ionising the separated analytes.

### FIELD

The present invention relates to paper spray mass spectrometry.

### BACKGROUND TO THE INVENTION

Paper spray mass spectrometry (PS-MS) continues to gain popularity as an analytical measurement technique, in part due to its disposable nature and simplicity, amongst other benefits. It has shown promising results for a variety of applications such as diagnostic biomarkers,¹ forensics,² water analysis,^{3, 4} drugs of abuse⁵⁻⁷ and therapeutic drug monitoring.⁸⁻¹⁰ One of the key advantages of PS-MS is its ability to provide fast and cost-effective analysis of mixtures directly while maintaining a sufficient level of specificity and sensitivity not far from that of LC-MS,¹¹ although usually lower. This should make PS-MS an attractive choice for use with clinical samples since it enables rapid testing without the need for expensive and time-consuming separation procedures. It also offers the potential for point-of-care (POC) analysis. PS-MS has been successfully used to analyse a range of biological matrices such as blood,^{6, 10, 12-18} urine,¹⁷ salivas,¹⁹ tears and milk,^{20, 21} demonstrating its potential for clinical research when coupled with portable mass spectrometry.

Yet despite its promise, PS-MS has not been incorporated into routine clinical practice. Various challenges remain, particularly achieving adequate quantitation precision in accordance with strict clinical requirements.²² This is likely due to the significant signal suppression caused by the matrix effect during ionisation, which is a challenge for ambient ionisation, in general, and especially when analysing complex biological matrices.^{15, 19, 21, 23-26}

Matrix interference occurs primarily during the ionisation of the analyte.²⁷ For clinical samples, endogenous substances such as cells, proteins, lipids and salts are the most common interferences.²⁷ When these substances co-elute with the target analyte, they can cause an ion suppression effect that can negatively impact quantitation. In our previous work, we found that direct analysis of paracetamol in saliva resulted in a low signal due to matrix interference, even after deproteination. Moreover, compared to a neat solution, the incomplete removal of the saliva matrix resulted in relatively poor spray stability.¹⁵ Matrix effect of PS-MS is the main problem that hinders its introduction into bedside monitoring.

A simpler, cheaper and faster technique with commensurate analytical performance, so as to be clinically viable, is needed.

### SUMMARY OF THE INVENTION

A first aspect provides a method comprising:
providing a sample comprising analytes, for example in a matrix, on a substrate, for example a monolithic substrate, comprising a stationary phase;
separating the analytes, for example mutually and/or relative to the matrix, on the substrate using a mobile phase; and
analysing the separated analytes using mass spectrometry, wherein the substrate provides, at least in part, an ion source for ionising the separated analytes.

A second aspect provides a method of Paper-Arrow Mass Spectrometry (PA-MS) comprising:
paper-chromatography (PC) and paper-spray mass-spectrometry (PS-MS) of a sample comprising analytes, for example using the same paper for the PC and for the PS-MS.

A third aspect provides an apparatus combining paper-chromatography (PC) and paper-spray mass-spectrometry (PS-MS).

A fourth aspect provides a method of selecting a mobile phase, for example for the method according to the first aspect, the method comprising:
providing a sample comprising a matrix, on a substrate, for example a monolithic substrate, comprising a stationary phase;
separating the matrix on the substrate in a direction using a first mobile phase;
dividing the substrate transverse, preferably orthogonally, to the direction;
providing analytes on the substrate, for example on each division of the divided substrate after dividing and/or before dividing; and
analysing the analytes using mass spectrometry, wherein the substrate provides, at least in part, an ion source for ionising the analytes, for example wherein each division of the divided substrate provides, at least in part, a respective ion source for ionising the analytes.

A fifth aspect provides a method of identifying a separation distance between analytes and a matrix, for example for the method according to the first aspect, the method comprising:
providing a sample comprising the analytes and the matrix, on a substrate, for example a monolithic substrate, comprising a stationary phase;
mutually separating the analytes and the matrix, on the substrate in a direction using a mobile phase;
dividing the substrate transverse, preferably orthogonally, to the direction; and
analysing the separated analytes using mass spectrometry, wherein the substrate provides, at least in part, an ion source for ionising the separated analytes, for example wherein each division of the divided substrate provides, at least in part, a respective ion source for ionising the separated analytes.

### DETAILED DESCRIPTION OF THE INVENTION

According to the present invention there is provided an apparatus, as set forth in the appended claims. Also provided is a method. Other features of the invention will be apparent from the dependent claims, and the description that follows.

### Method

The first aspect provides a method comprising:
providing a sample comprising analytes, for example in a matrix, on a substrate, for example a monolithic substrate, comprising a stationary phase;
separating the analytes, for example mutually and/or relative to the matrix, on the substrate using a mobile phase; and
analysing the separated analytes using mass spectrometry, wherein the substrate provides, at least in part, an ion source for ionising the separated analytes.

In this way, the analytes are separated, for example mutually and/or relative to the matrix, on the substrate using the mobile phase and the separated analytes are analysed using mass spectrometry. Particularly, the ion source for ionising the separated analytes on the substrate is provided, at least in part, by the substrate i.e. the same substrate comprising the stationary phase for separating the analytes using the mobile phase. In this way, analyte separation and mass spectrometry of the separated analytes is simpler, cheaper and/or faster than conventional techniques since the same substrate is used for both analyte separation and mass spectrometry of the separated analytes. For example, filter paper may be used as the substrate, wherein separating the analytes, for example mutually and/or relative to the matrix, on the substrate using the mobile phase comprises and/or is paper chromatography and/or solid phase extraction; and wherein analysing the separated analytes using mass spectrometry comprises and/or is paper spray mass spectrometry. That is, the method according to the first aspect may comprise paper-chromatography (PC) and paper-spray mass-spectrometry (PS-MS) of the sample comprising the analytes, for example using the same paper for the PC and for the PS-MS. In this way, the analytes may be separated from the matrix, for example, and the separated analytes analysed using mass spectrometry, wherein matrix effects (for example, suppression and/or interference) due to the matrix during the mass spectrometry are relatively reduced, compared with mass spectrometry of the analytes without separation, for example in the matrix. In this way, limits of detection and/or limits of quantification may be relatively lowered, thereby improving decision making, for example clinical decision making.

In more detail, aiming to solve the matrix effect of PS-MS without compromising its convenience, the inventors have designed a novel approach utilising a paper arrow, to seamlessly combine a short process of paper chromatography with PS-MS in a unique and complementary manner.²⁸ This technique, named as paper-arrow mass spectrometry (PA-MS) (previously named Paper-Chromatography-Spray Mass Spectrometry (PCS-MS) in previous first applications from which priority is claimed), combines sample collection, extraction, enrichment, separation and ionisation onto a single paper strip, and the entire analysis process, from sample to result, can be carried out within a few minutes with only a small quantity of raw liquid media. For example, paracetamol analysis was completed within 10 minutes after application of 2 μL of raw human saliva.

The data shows that PA-MS can easily treat biofluid samples of saliva, urine, sweat, and plasma to a satisfactory level at bedside to pave the way for portable MS analysis, which can be very useful in various clinical scenarios, e.g. (1) overdose and abuse in A&E, (2) adherence and compliance with treatments of chronic diseases, and (3) model-informed precision dosing.

To demonstrate PA-MS's development and validation, paracetamol is chosen as an exemplar analyte. Paracetamol (acetaminophen) is commonly used as a painkiller throughout the world without prescription.²⁹ Suspected overdose of paracetamol is a common reason to visit Emergency Departments.³⁰ Annually in the UK, approximately 50,000 acute hospital admissions were due to the hepatotoxicity of overdosed paracetamol. In such cases, the concentration of paracetamol should be monitored to determine whether the antidote acetylcysteine is needed as well as the treatment dose and duration. Thus, rapidly quantifying paracetamol concentration is crucial for clinical decision-making. However, this critical need has not yet been previously met.

The PA-MS method requires only 2 μL of human saliva sample and ˜5-min PC separation without any sample pre-treatment, prior to MS analysis. The absorbent substrate is a short chromatography paper arrow with one triangular end. Prior to spraying/ionisation, a 2 μL sample is added and dried on the flat end of the paper arrow. Then the flat end of the paper arrow is dipped into a specified solvent mixture. The solvent carries the analyte up to the paper triangular tip, separating the analyte from the matrix. Finally, the analyte is isolated by removing (e.g. cutting) the triangular tip from the paper arrow for a direct MS analysis. The entire process is simple and can be readily completed within a few minutes, yielding excellent analytical performance (LOD: 61 ng/mL, LOQ: 185 ng/ml, r²: 0.9998, accuracy<5.4%, and CV<6.4%). These results using PA-MS were further compared against the current state-of-the-art, LC-MS/MS and PA-MS performed equally well.

### Method

In one example, the method comprises paper-chromatography (PC) and paper-spray mass-spectrometry (PS-MS) of the sample comprising the analytes, for example using the same paper for the PC and for the PS-MS.

In one example, the substrate comprises and/or is paper, for example a strip (such as a rectangular strip) or an arrow (such as a rectangular strip having an arrow head, for example a triangle, at one end). In one example, providing the sample comprising the analytes, for example in a matrix, on the substrate comprising the stationary phase comprises and/or is providing the sample comprising the analytes, for example in a matrix, on the paper. In one example, separating the analytes, for example mutually and/or relative to the matrix, on the substrate using the mobile phase comprises and/or is paper chromatography comprising separating the analytes, for example mutually and/or relative to the matrix, on the paper using the mobile phase. In one example, analysing the separated analytes using mass spectrometry comprises paper spray mass spectrometry, wherein the paper, for example an apex thereof, provides, at least in part, the ion source for ionising the separated analytes.

### Sample

The method comprises providing the sample comprising the analytes, for example in a matrix, on the substrate, for example at an origin such as a predefined origin thereupon.

In one example, the analytes comprise: an active pharmaceutical ingredient and/or a metabolite thereof; a narcotic (also known as a drug of abuse) and/or a metabolite thereof; an endogenous compound; a xenobiotic; a biomolecule; a toxic compound; an environmental contaminant; a pesticide; a residue; an explosive.

In one example, the active pharmaceutical ingredient and/or a metabolite thereof comprises paracetamol and/or a metabolite, for example N-acetyl-p-benzoquinone imine (NAPQI), thereof. In this way, the method according to the first aspect may be used for non-invasive diagnosis of paracetamol overdose, for example using a saliva sample.

In one example, the active pharmaceutical ingredient and/or a metabolite thereof comprises an active pharmaceutical ingredient that may induce liver injury even under therapeutic dosage, for example an antituberculotic medicine, an antifungal and/or an antiepileptic. In this way, the method according to the first aspect may be used for non-invasive diagnosis of Drug-Induced Liver Injury (DILI), for example using a saliva sample.

In one example, the narcotic comprises and/or is marijuana, cannabidiol oil and/or a synthetic cannabinoid (SC). Synthetic cannabinoids are synthetically manufactured compounds that mimic the active ingredient of marijuana tetrahydrocannabinol (THC) in structure or function. In one example, the metabolite thereof comprises and/or is tetrahydroannabinol (THC-COOH). In this way, the method according to the first aspect may be used for non-invasive diagnosis of overdose, for example using a urine sample.

In one example, the narcotic comprises and/or is a new psychoactive substances (NPS), also called synthetic or designer drugs, are similar to historically more common drugs in structure or function, for example fentanyl and/or a fentanyl analogue. In this way, the method according to the first aspect may be used for non-invasive diagnosis of overdose, for example using a urine sample.

In one example, the endogenous compound comprises and/or is bilirubin (conjugated and/or unconjugated) and/or one or more bile salts (a group of bile acids). In this way, the method according to the first aspect may be used for non-invasive diagnosis of drug-induced liver injury (DILI), for example using a urine sample.

In one example, the sample comprises a biological sample, such as saliva, urine, whole blood and/or serum; an environmental sample, such as water, leachate. That is, the matrix may be relatively complex and/or the analytes may be present at relatively low concentrations.

Generally, biological samples (also known as biofluids) have complex matrices, including relatively high concentrations of salts and/or biomolecules, that may adversely affect mass spectrometry analysis of the analytes, due to matrix effects such as suppression and/or interference. By separating the analytes relative to the matrix (i.e. by mutually separating the analytes and the matrix) on the substrate before mass spectrometry using the same substrate, the matrix effects are relatively reduced, as described previously, while analysis of the analytes is simpler, cheaper and/or faster than conventional techniques.

In one example, providing the sample comprising the analytes on the substrate comprises providing the sample comprising the analytes on the substrate in a volume in a range from 0.1 μl to 100 μl, preferably in a range from 0.5 μl to 50 μl, more preferably in a range from 1 μl to 10 μl, for example 1 μl, 2 μl, 3 μl, 4 μl, 5 μl, 6 μl, 7 μl, 8 μl, 9 μl or 10 μl. In this way, only a relatively small volume of sample is required.

### Substrate

The method comprises providing the sample comprising the analytes on the substrate, for example a monolithic (i.e. single piece) substrate, comprising the stationary phase. In one example, the substrate provides the stationary phase. In one example, the substrate is the stationary phase.

It should be understood that the same substrate is used for both analyte separation and mass spectrometry of the separated analytes.

In one example, the substrate comprises and/or is a porous substrate. In one example, the substrate comprises and/or is a fibrous substrate, for example a cellulosic substrate such as paper.

In one example, the substrate, for example the porous substrate and/or the fibrous substrate, comprises and/or is paper, for example filter paper, chromatographic paper, or any other porous, water-wettable material. In one example, the substrate, for example the porous substrate and/or the fibrous substrate, has a thickness in a range from about 10 μm to about 700 μm, preferably in a range from about 150 μm to about 200 μm.

In one example, the substrate, for example the porous substrate and/or the fibrous substrate, comprises and/or is cellulose filter paper, ashless filter paper, nitrocellulose filter paper, a glass microfiber filter, porous polyethylene sheets, polyvinylidene difluoride (PVDF) paper, or chromatography paper. Other porous materials are also considered such as flat materials coated with a layer of absorbent material such as silica gel, cellulose powder, or alumina oxide. In one example, the substrate, for example the porous substrate and/or the fibrous substrate, comprises and/or is a general-purpose cellulose filter paper, a qualitative cellulose filter paper, a quantitative ashless cellulose filter paper, a quantitative hardened ashless cellulose filter paper, or a wet strengthened filter paper. Examples of general-purpose cellulose filter papers include, but are not limited to Grade 0858, Grade 0903, Grade 201 qualitative, Grade 202 qualitative, Grade 226 qualitative, Grade 2589 A, and Grade 520 a filter papers. Examples of qualitative cellulose filter papers include, but are not limited to Grade 1, Grade 2, Grade 3, Grade 4, Grade 5, Grade 6, Grade 588, Grade 591, Grade 595, Grade 597, Grade 597 L, Grade 598, Grade 602 h, and Grade 602EH qualitative filter papers. Examples of quantitative hardened ashless cellulose filter papers include, but are not limited to Grade 589/3, Grade 40, Grade 41, Grade 42, Grade 43, and Grade 44 quantitative ashless filter papers. Examples of quantitative hardened ashless cellulose filter papers include, but are not limited to Grade 540, Grade 541, and Grade 542 hardened ashless cellulose filter papers. Examples of wet strengthened cellulose filter papers include, but are not limited to Grade 113, Grade 114, Grade 1573, Grade 1575, Grade 91, and Grade 93 qualitative wet strengthened filter papers. Examples of chromatographic paper includes, but are not limited to Grade 1 Chr, Grade 17 Chr, Grade 2 Chr, Grade 20 Chr, Grade 2668 Chr, Grade 2727 Chr, Grade 3 Chr, Grade 31ET Chr, Grade 3 MM, Grade 4 Chr, and Grade 54 SFC cellulose chromatography papers. In certain embodiments, the porous material is Grade 31ET Chr cellulose chromatography paper. In other embodiments, the porous material is Grade 3MM Chr cellulose chromatography paper. The filter papers provided as examples above are Whatman filters, available from GE Healthcare Lifesciences, although filter papers from other manufacturers having similar properties to those listed above are also suitable.

The skilled person is able to select an appropriate porous material for use in the methods described herein. Parameters that influence the substrate's effectiveness and appropriateness for a particular use include, but are not limited, to pore size and particulate retention, adsorption, pH, surface properties, thickness, and wet strength, as understood by the skilled person.

In one example, the substrate comprises and/or is a non-porous substrate.

In one example, the substrate comprises and/or is a layered substrate, for example having porous, fibrous and/or non-porous layers.

In one example, the substrate comprises and/or is a thin layer chromatography (TLC) plate (for example, having a porous layer overlaying a non-porous layer i.e. a layered substrate) or a TLC material. Suitable TLC materials include silica gel, Diol-bonded silica gel, for example for HPTLC plates and aluminium oxide. Other suitable TLC materials are known.

Generally, the stationary phase for TLC is typically organic or inorganic thin layer, such as silica, alkyl-silica (C8 or C18), cellulose, and monolithic polymer coated on metal, plastic, or glass sheets; the mobile phase for TLC analysis is typically organic solutions spanning a wide range of hydrophobicities. During the separation, the edge of the TLC plate is immersed in the mobile phase, which is developed through capillary force. The diversity of interactive forces among the analytes, mobile phase, and stationary phase cause different analytes to move at different rates on the TLC plate. The separation of the chemical compounds on a TLC plate is quantified in terms of the value of the retention factor Rf (distance of analyte migration/distance of mobile phase migration).

### Separating the Analytes

The method comprises separating the analytes, for example mutually and/or relative to the matrix, on the substrate using the mobile phase. It should be understood that the analytes and/or the matrix are separated spatially, since the analytes and/or the matrix move at relatively different rates along the substrate, for example due to a diversity of interactive forces among the analytes, the matrix, the mobile phase and the stationary phase. In one example, the mobile phase and/or the stationary phase are selected (for example, screened) whereby a rate of movement along the substrate of the analytes is relatively greater than a rate of movement along the substrate of the matrix. That is, the retention factor Rf for the analytes is greater than the retention factor Rf for the matrix. In this way, the analytes move relatively faster than the matrix and are mutually spatially separated therefrom and downstream (i.e. ahead) thereof.

In one example, the separating of the analytes on the substrate using the mobile phase comprises and/or is paper chromatography (PC); and/or solid phase extraction (SPE). PC and SPE are known. While PC is generally used to separate compounds having different colours, PC may be used to separate the analytes from the matrix, in absence of colour and/or without staining, and subsequently, the separated analytes may be analysed using mass spectrometry, relatively free from matrix effects. SPE provides extraction of the analytes from the matrix, thereby cleaning up, purifying and/or pre-concentrating the analytes prior to analysis using mass spectrometry, relatively free from matrix effects. In one example, the SPE comprises and/or is normal phase SPE. In one example, the SPE comprises and/or is reverse phase SPE.

In one example, the mobile phase comprises a proton acceptor, a proton donor and/or a dipole. In general, every mobile phase has its own selective properties. L. R. Snyder and J. J. Kirkland investigated and compared various mobile phases and grouped those with similar effects together into selectivity groups. The selectivity groups are organized into a selectivity triangle that makes it possible to visually compare mobile phases. The most important practical consequence of the selectivity triangle is that if a certain mobile phase cannot provide sufficient selectivity in a given separation, it is unlikely that any other mobile phase in the same group can do so. Instead, you should use mobile phase from other selectivity groups. Selecting a suitable mobile phase is understood by the skilled person.

In one example, separating the analytes, for example mutually and/or relative to the matrix, on the substrate using the mobile phase comprises and/or is one-dimensional separation, for example one-dimensional PC and/or one-dimensional SPE. In one example, separating the analytes, for example mutually and/or relative to the matrix, on the substrate using the mobile phase excludes (i.e. does not include) two-dimensional separation, for example two-dimensional PC and/or two-dimensional SPE. In this way, analyte separation is simpler, cheaper and/or faster since separating the analytes comprises and/or is one-dimensional separation c.f. two-dimensional separation.

### Mass Spectrometry

The method comprises analysing the separated analytes using mass spectrometry, wherein the substrate provides, at least in part, the ion source for ionising the separated analytes.

It should be understood that the same substrate is used for both analyte separation and mass spectrometry of the separated analytes. It should be understood that the step of analysing the separated analytes using mass spectrometry is after (i.e. subsequent to) separating the analytes, for example mutually and/or relative to the matrix, on the substrate using the mobile phase. It should be understood that the substrate provides at least in part the ion source (i.e. a part of the ion source, for example a physical component thereof such as a tip or an emitter tip) for ionising the separated analytes

In one example, the method comprises washing (and optionally drying) the substrate before the step of providing the sample comprising analytes, for example in a matrix, on the substrate. In one example, the method comprises drying the substrate before the step of analysing the separated analytes using mass spectrometry, for example to remove the mobile phase used for the separating. In one example, the method comprises rinsing the substrate before the step of analysing the separated analytes using mass spectrometry.

In one example, the mass spectrometry comprises and/or is: paper spray mass spectrometry; leaf spray mass spectrometry; coated blade spray mass spectrometry; and/or solid-substrate electrospray. Paper spray mass spectrometry, leaf spray mass spectrometry, coated blade spray mass spectrometry and solid-substrate electrospray are known. Other suitable mass spectrometry includes matrix-assisted laser desorption/ionization (MALDI) MS, ambient ionization MS including desorption electrospray ionization (DESI) MS, direct analysis in real time (DART) MS, desorption atmospheric pressure chemical ionization (DAPCI) MS and liquid extraction surface analysis (LESA).

In one example, the mass spectrometry comprises using a solvent, for example a spray solvent. Generally, a solvent may be used to improvement movement of the analytes along the substrate, for example along the provided, at least in part, ion source. For example, by dissolving the analytes in the solvent, migration thereof along the substrate under an applied electrical potential may be improved. Generally, the solvent is different from the mobile phase. Suitable solvents are known. In one example, the solvent is selected to reduce, for example minimise, adducting of the analytes, for example metal ion adducting of the ions.

In one example, analysing the separated analytes using mass spectrometry comprises applying an electric potential to the substrate, thereby providing, at least in part, the ion source for ionising the separated analytes. In this way,

In one example, the substrate comprises a protrusion, for example having an apex, thereby providing, at least in part, the ion source, such as a tip or an emitter tip, for ionising the separated analytes. Suitable protrusion geometries are known and may be provided by cutting the substrate, for example, before or after the step of providing the sample comprising analytes, for example in a matrix, on the substrate or before or after the step of separating the analytes, for example mutually and/or relative to the matrix, on the substrate using the mobile phase.

In one example, the substrate comprises a plurality of protrusions, for example each having an apex, thereby providing, at least in part, a plurality of ion sources for ionising the separated analytes, for example as described previously.

In one example, the substrate has a first end and a mutually-opposed second end, defining an axis (for example a longitudinal axis) therebetween, wherein separating the analytes on the substrate using the mobile phase comprises separating the analytes axially, for example one-dimensional separation. In this way, the analytes are separated spatially, for example from the matrix, along the axis of the substrate.

In one example, separating the analytes on the substrate using the mobile phase comprises contacting the substrate with the mobile phase proximal and/or at the first end of the substrate, for example by immersing the first end of the substrate in the mobile phase and/or supplying the mobile phase on the substrate proximal and/or at the first end of the substrate. In this way, analyte separation is simpler, cheaper and/or faster since the substrate may be readily immersed into the mobile phase and/or the mobile phase readily supplied thereon, for example using a pipette.

In one example, the second end of the substrate provides, at least in part, the ion source for ionising the separated analytes. In one example, the second end of the substrate comprises a protrusion, for example having an apex, thereby providing, at least in part, the ion source, such as a tip or an emitter tip, for ionising the separated analytes, as described previously.

In one example, the method comprises parting the substrate, for example into a first part and a second part, after separating the analytes, for example mutually and/or relative to the matrix, on the substrate using the mobile phase, for example wherein the second part provides, at least in part, the ion source for ionising the separated analytes. In this way, the matrix may be retained on the first part of the substrate, for example, while the separated analytes are on the second part of the substrate. In this way, analysis of the separated analytes using mass spectrometry is improved since matrix effects (for example, suppression and/or interference) due to the matrix during the mass spectrometry are relatively reduced since the matrix is retained on the first part of the substrate, which does not provide, at least in part, the ion source. It should be understood that the first part may be discarded. In one example, parting the substrate, for example into the first part and the second part, after separating the analytes comprises shearing, cleaving and/or cutting the substrate, for example between the separated analytes and the matrix. In one example, parting the substrate, for example into the first part and the second part, after separating the analytes comprises parting the substrate, for example into the first part and the second part, at a predefined distance from the origin. In this way, mass spectrometry of the separated analytes is simpler, cheaper and/or faster since the substrate may be readily parted at a predefined distance from the origin. In one example, the substrate comprises and/or provides an indicator for indicating the origin and/or a guide for guiding the parting. In one example, parting the substrate, for example into a first part and a second part, after separating the analytes comprise parting the substrate, for example into a first part and a second part, after separating the analytes transverse, for example orthogonal to, the axis. In one example, parting the substrate, for example into a first part and a second part, after separating the analytes comprises parting a protrusion, for example having an apex, from the substrate.

In one example, the substrate is symmetric about the axis (i.e. wherein the axis defines a mirror line or a mirror plane). In this way, movement, for example wicking, of the mobile phase and hence movement, for example transport, of the analytes is relatively uniform along the substrate. In one example, the substrate excludes (i.e. does not include) asymmetric features, for example edge features, and/or perforations. In this way, movement, for example wicking, of the mobile phase and hence movement, for example transport, of the analytes is relatively uniform along the substrate.

In one example, the substrate comprises and/or is a strip (such as a rectangular strip) or an arrow (such as a strip, for example a rectangular strip, having an arrow head, for example a triangle, (i.e. a protrusion, for example having an apex) at one end) i.e. symmetric about the axis. In one example, parting the substrate, for example into a first part and a second part, after separating the analytes comprises parting the arrow head (more generally, a protrusion, for example having an apex) from the substrate. In one example, a base of the triangle is equal to a width of the strip. In one example, a base of the triangle is greater than a width of the strip. In one example, a distance from the origin to the base of the triangle is about equal to (for example, within 50%, preferably within 25%, more preferably within 10%) or is equal to a height of the triangle. In one example, an area of the strip between the origin and the base of the triangle is about equal to (for example, within 50%, preferably within 25%, more preferably within 10%) or is equal to an area of the triangle. In one example, the triangle is an equilateral triangle or an isosceles triangle.

In one example, separating the analytes, for example mutually and/or relative to the matrix, on the substrate using the mobile phase comprises and/or is one-dimensional separation, for example one-dimensional PC and/or one-dimensional SPE. In one example, separating the analytes, for example mutually and/or relative to the matrix, on the substrate using the mobile phase excludes (i.e. does not include) two-dimensional separation, for example two-dimensional PC and/or two-dimensional SPE. In this way, analyte separation is simpler, cheaper and/or faster since separating the analytes comprises and/or is one-dimensional separation c.f. two-dimensional separation.

### Paper-Arrow Mass Spectrometry

The second aspect provides a method of Paper-Arrow Mass Spectrometry (PA-MS) comprising: paper-chromatography (PC) and paper-spray mass-spectrometry (PS-MS) of a sample comprising analytes, for example using the same paper for the PC and for the PS-MS.

The PC, the PS-MS, the sample, the analytes and/or the paper may be as described with respect to the first aspect. The method according to the second aspect may include any step as described with respect to the first aspect.

The third aspect provides an apparatus combining paper-chromatography (PC) and paper-spray mass-spectrometry (PS-MS).

The PC, the PS-MS, the sample, the analytes and/or the paper may be as described with respect to the first aspect and/or the second aspect. The apparatus according to the second aspect may include any feature as described with respect to the first aspect.

### Selecting a Mobile Phase

The fourth aspect provides a method of selecting a mobile phase, for example for the method according to the first aspect, the method comprising:
providing a sample comprising a matrix, on a substrate, for example a monolithic substrate, comprising a stationary phase;
separating the matrix on the substrate in a direction using a first mobile phase;
dividing the substrate transverse, preferably orthogonally, to the direction;
providing analytes on the substrate, for example on each division of the divided substrate after dividing and/or before dividing; and
analysing the analytes using mass spectrometry, wherein the substrate provides, at least in part, an ion source for ionising the analytes, for example wherein each division of the divided substrate provides, at least in part, a respective ion source for ionising the analytes.

The mobile phase, the sample, the matrix, the substrate, the stationary phase, the separating, the analytes, the analysing, the mass spectrometry and/or the ion source may be as described with respect to the first aspect.

In one example, the method comprises repeating the steps using a second mobile phase and selecting the first mobile phase or the second mobile phase based, at least in part, on matrix effects, adducting, signal intensity (height or area) and/or sensitivity (i.e. signal intensity to noise ratio) during the mass spectrometry, for example the particular mobile phase resulting in relatively lower matrix effects, relatively lower adducting, relatively higher signal intensity and/or relatively higher sensitivity during the mass spectrometry.

In one example, the method according to the first aspect comprises the method according to the fourth aspect.

### Identifying a Separation Distance

The fifth aspect provides a method of identifying a separation distance between analytes and a matrix, for example for the method according to the first aspect, the method comprising:
providing a sample comprising the analytes and the matrix, on a substrate, for example a monolithic substrate, comprising a stationary phase;
mutually separating the analytes and the matrix, on the substrate in a direction using a mobile phase;
dividing the substrate transverse, preferably orthogonally, to the direction; and
analysing the separated analytes using mass spectrometry, wherein the substrate provides, at least in part, an ion source for ionising the separated analytes, for example wherein each division of the divided substrate provides, at least in part, a respective ion source for ionising the separated analytes.

The mobile phase, the sample, the matrix, the substrate, the stationary phase, the separating, the analytes, the analysing, the mass spectrometry and/or the ion source may be as described with respect to the first aspect.

In one example, the method comprises identifying the separation distance based, at least in part, matrix effects, adducting, signal intensity (height or area) and/or sensitivity (i.e. signal intensity to noise ratio) during the mass spectrometry of the divisions of the divided substrate, for example the particular division of the divided substrate having relatively lower matrix effects, relatively lower adducting, relatively higher signal intensity and/or relatively higher sensitivity.

In one example, the method according to the first aspect comprises the method according to the fifth aspect.

### Definitions

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention, such as colourants, and the like.

The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

### BRIEF DESCRIPTION OF THE DRAWINGS

For a better understanding of the invention, and to show how exemplary embodiments of the same may be brought into effect, reference will be made, by way of example only, to the accompanying diagrammatic Figures, in which:

FIG. 1 The figure depicts the whole process of PA-MS analysis with a raw saliva sample. 2 μL of saliva is first applied to a paper arrow, which is then introduced into the mobile phase via the base of the arrow's shaft. The analytes in the biofluid are separated and concentrated at the tip of the arrowhead through a ˜5-min process of PC. Finally, the arrowhead is cut off for PS-MS analysis.

FIG. 2 Estimation of the positions of saliva components after PC. (a) A schematic workflow illustrating the PC method, where 2 μL of blank saliva was treated with the mobile phase for 12 min. The paper strip was cut along the serrated regions (Regions 0-10). 2 μL of water spiked with 100 μg/mL paracetamol was pipetted onto each piece of paper (i.e. each region). After they were air-dried, MS analysis was performed. (b) Signal suppression extent of [M+H]⁺ of paracetamol at each region in saliva matrix after PC. It was compared with that of blank water spiked with the same concentration of paracetamol, which was directly added onto an independent piece of paper region without saliva and PC (mean±SD, n=3). (c) SIM showing the intensity of [M+Na]⁺ and [M+K]⁺ ions of paracetamol in each region (mean±SD, n=3).

FIG. 3 Identification of paracetamol's position after PC. (a) A schematic diagram of the workflow. 2 μL of saliva, spiked with 100 μg/mL of paracetamol was added at Region 0. It was inserted into the mobile phase for 12 min of PC. After being dried in the air, it was cut and each piece of regions was used for MS analysis (Supplementary Method 3.1). (b) Intensities of [M+H]⁺, [M+Na]⁺, [M+K]⁺ of paracetamol in each region (mean±SD, n=3).

FIG. 4 The figure presents a comparison of three positions of sample application on an arrow-shaped paper substrate. (a) Schematic presentation of the experiment in which 2 μL of 100 μg/mL paracetamol in saliva was spotted onto application points located at distances of 5 mm, 10 mm and 15 mm from the base of the arrowhead. After drying in air, the arrow-shaped paper strips were immersed in the mobile phase, and the front of the mobile phase reached the tip of the arrowhead within 5 min. (b) Intensities of [M+H]⁺ (mean±SD, n =4). One-way ANOVA showed p=0.0032, Tukey's post-hoc test was noted in the figure (**p<0.01).

FIG. 5 Comparison between PA-MS and PS-MS. (a) The intensities of [M+H]⁺, [M+Na]⁺ and [M+K]⁺ for 2 μL 100 μg/mL paracetamol in water or saliva, measured by PA-MS or PS-MS (mean±SD, n =3). (b) Staining results, illustrating the movement of paracetamol from water and saliva on triangular paper after PS-MS, and on arrow-shaped paper after PA-MS. For this analysis, 2 μL of 100 μg/mL paracetamol in water or saliva was applied and stained by the colourimetric method (Supplementary method 6). (c) Comparison of participants' resting saliva samples and blank water, each spiked with 100 μg/mL paracetamol, and detected by PA-MS and PS-MS (mean±SD, n =3).

FIG. 6 Calibration curve of salivary paracetamol detected by PA-MS/MS with a concentration range of 0.2-200 μg/mL, including the timed British clinical overdose treatment reference concentrations. Data expressed as mean±SD, n=5.

FIG. 7 Matrix effects of paracetamol and paracetamol-D4 (IS) by PA-MS/MS

FIG. 8 is a graph of peak intensity of daughter ion for different matrices.

FIG. 9 Lin's concordance correlation of the three testing sets with gold standard set. (a) Correlation of plasma paracetamol concentrations measured by PA-MS with that by the gold standard set. (b) Correlation of resting saliva paracetamol concentrations measured by PA-MS with the gold standard set. Two abnormal values were circled out. (c) Correlation of resting saliva paracetamol concentrations measured by PA-MS with the gold standard set (excluding the 2 circled abnormal values). (d) Correlation of stimulated saliva paracetamol concentrations measured by PA-MS with the gold standard set. One abnormal value was circled out. (e) Correlation of stimulated saliva paracetamol concentrations measured by PA-MS with the gold standard set (excluding the circled abnormal value).

FIG. 10 Bland-Altman difference plots of the three testing sets with gold standard set. (a) Bland-Altman difference plots of plasma paracetamol concentrations measured by PA-MS with that by the gold standard set. (b) Bland-Altman difference plots of resting saliva paracetamol concentrations measured by PA-MS with the gold standard set. (c) Linear regression analysis between differences and means of resting saliva measured by PA-MS and the gold standard set. (d) Bland-Altman difference plots of stimulated saliva paracetamol concentrations measured by PA-MS with the gold standard set.

FIG. 11 Time curves of ratios of the three testing sets over gold standard set.

FIG. 12 Differences between PA-MS with previous published methods (1): sample application for analytes (e.g. A, B, C) mixed in one biofluid. This figure schematically depicts a comparison of known methods of applying analytes to a substrate with the method of applying analytes to a substrate of the present invention. C1 refers to the method patented as: a paper chromatography-mass spectrometry analysis and detection device.³¹ C2 refers to the novel paper-based microfluidic cassette for 2D paper chromatography and PS-MS.³²

FIG. 13 Differences between PA-MS with previous published methods (2): paper chromatography (PC) after sample application. In C1 and C2, PC was done with the same mobile phase, and stops before the solvent front reaches the far end of the paper strip.

This is standard for paper chromatography methods. While in PA-MS, for each analyte, PC is done separately, each with specifically chosen mobile phases. And the mobile phase is allowed to reach the far end of the paper strip. The solvent front reaching the end of the arrow is important. Moreover, the PC flow direction is in the same direction as the subsequent ‘electrospray’.

FIG. 14 Differences between PA-MS with previous published methods (3): distribution of analytes A, B, and C after PC. C1 and C2 partially separate analytes with unavoidable diffusion along PC direction. Comparatively, in PA-MS, analytes are efficiently separated/extracted by the specific mobile phases and concentrated close to the tip of the arrowhead. The issue with diffusion which leads to reduced sensitivity for C1 and C2 is completely circumvented.

FIG. 15 Differences between PA-MS with previous published methods (4): preparation for Spray. This figure schematically depicts known methods of preparing a substrate for spray with the method presented herein. In C1, triangles were cut off for paper spray with huge loss of analytes that have diffused out of the triangular area. In C2, second PC was done orthogonal with the first PC for enrichment of analytes, but analytes are lost too at both sides of tips. By cutting off the arrow head, no analytes are lost at all in PA-MS.

FIG. 16 Differences between PA-MS with previous published methods (5): Spray directions. C1, spray direction is orthogonal to PC direction; in C2, spray direction is parallel to 2nd PC direction, but orthogonal to the 1st PC direction. While in PA-MS, spray direction is parallel to PC direction. schematically depicts known methods of spray after paper chromatography with the method presented herein.

FIG. 17 Differences between PA-MS with previous published methods (6): a Proof of the advantage of PA-MS over 2D PC method. Lin et al detected acetaminophen (also called paracetamol) with different concentration levels by their 2D PC method. As shown in FIG. 17 (A), 105 ppb (100 ug/mL) acetaminophen's ion intensity only reached a level of 10^{7,33}. Coincidently, we also detected acetaminophen with PA-MS at the same concentration level. Without any loss of analytes, the signal intensity was 20 times higher than 2D-PC-method (B).²⁸

FIG. S1 schematically depicts substrates according to an exemplary embodiment. In more detail, FIG. S1 shows measures (i.e. dimensions) of three shapes of filter paper: (a) measures of serrated paper strips used to identify retention factors of salivary components and analyte of paracetamol, (b) measures of triangle paper used for traditional PS-MS, and (c) measures of arrow-shaped paper strips used for PA-MS.

FIG. S2 shows process and result of mobile phase selection for PA-MS, according to an exemplary embodiment: (a) process of mobile phase screening for PA-MS, (b) intensity of [M+H]⁺ after PC with pure ethyl acetate, (c) mass spectrum of full scan at the origin after PC with ethyl acetate, and (d) intensities of [M+H]⁺ after PC with 3 mobile phases and the intensities of traditional PS-MS.

FIG. S3 shows mass spectra of full scan of each region from 0-10. 2 μL blank saliva was applied and 12 min of PC was done with 50 mM NH₄HCO₂ in 9:1 ethyl acetate: formic acid (v/v). After then, regions of 0-10 were cut apart, and 2 μL 100 μg/mL paracetamol in deionised water was applied onto each piece of paper. After drying in air, mass spectrometry was conducted. The peaks corresponding to [M+H]⁺, [M+Na]⁺, [M+K]⁺ were monitored.

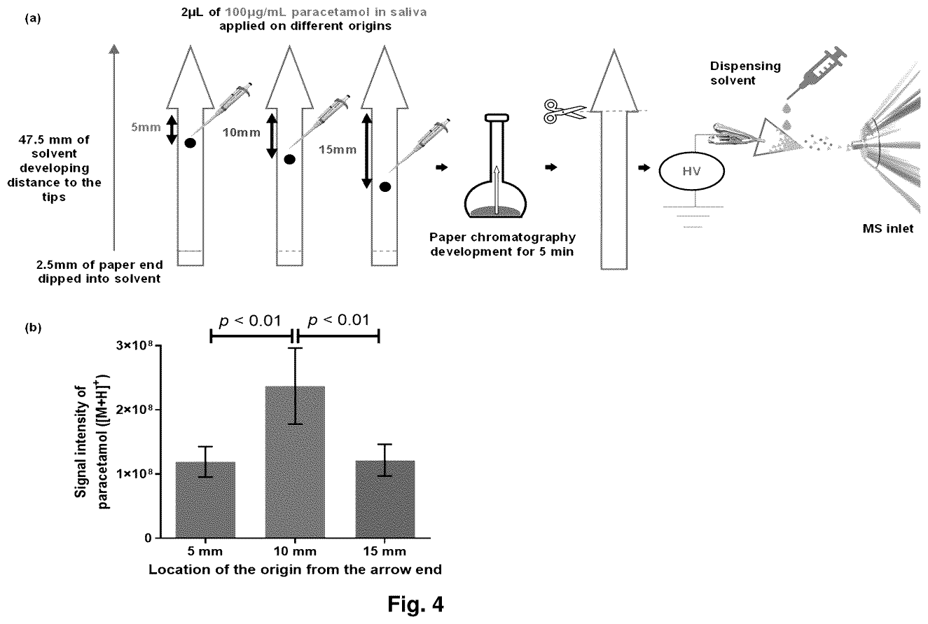

FIG. S4 shows SIM Chromatograms of [M+H]⁺. 2 μL 100 μg/mL paracetamol in saliva or water was applied onto triangle paper or arrow-shaped paper. Triangle paper was detected by PS-MS, while arrow-shaped paper underwent PA-MS as seen in FIG. 4(a).

FIG. S5 Staining results. 2 μL of a sample was applied onto the site marked by a pencil: (a) blank water, (b) blank saliva, (c) blank water spiked with 100 μg/mL paracetamol, (d) saliva spiked with 100 μg/mL paracetamol. This was stained with Prussian Blue spray as described in Experimental Section 2.2.3, (1) without or (2) after 5-min PC development.

FIG. S6 shows comparison of calibration curves of paracetamol detection. (a) Calibration curve of paracetamol in raw saliva detected by PA-MS/MS. (b) Calibration curve of paracetamol in raw saliva detected by PS-MS/MS. (c) Calibration curve of paracetamol in pure water detected by PA-MS/MS. (d) Calibration curve of paracetamol in pre-treated saliva detected by UPLC-MS/MS. (e) Calibration curve of paracetamol in pre-treated saliva detected by PS-MS/MS.

FIG. S7 shows experiment process used to evaluate the analysis of paracetamol in saliva by PA-MS/MS, according to an exemplary embodiment.

FIG. S8 Sample collection process.

FIG. S9 The figure depicts the whole process of PA-MS analysis with a raw saliva sample.

### DETAILED DESCRIPTION OF THE DRAWINGS

Described herein, by way of non-limiting example, is Paper-Chroma-Spray Ionisation Mass Spectrometry as a novel approach to detect paracetamol in saliva, according to the first aspect which provides a method comprising:
providing a sample (in this example, saliva) comprising analytes (in this example, paracetamol), for example in a matrix (in this example, saliva), on a substrate (in this example, paper), for example a monolithic substrate, comprising a stationary phase (in this example, paper);
separating (in this example, paper chromatography) the analytes, for example mutually and/or relative to the matrix, on the substrate using a mobile phase (in this example, 50 mM ammonium formate in 9:1 ethyl acetate: formic acid (v/v)); and
analysing the separated analytes using mass spectrometry (in this example, paper spray mass spectrometry), wherein the substrate provides, at least in part, an ion source (in this example, a triangular emitter tip for electrospray) for ionising the separated analytes.

To develop the method of PA-MS, a serrated paper strip was designed to optimise PC separation by screening mobile phases and to identify the location of paracetamol after PC. In short, 2 μL of 100 μg/mL paracetamol in saliva was applied to the origin and 12 min of PC was done with different mobile phases. When the front of the mobile phase reached the 10^{th} region from the origin during the PC, the paper was then allowed to dry in air for 1 min. Then the regions of 0-10 were cut apart manually and paracetamol adducts of [M+H]⁺ on each piece were detected by positive mode of MS. For traditional PS-MS, 0.2 μL of 100 μg/mL paracetamol in water or saliva was added onto the triangle paper and was detected by traditional PS-MS after being dried in air.

From the data generated the peak intensities of [M+H]⁺, [M+Na]⁺, and [M+K]⁺ were monitored. Eventually, 50 mM ammonium formate in 9:1 ethyl acetate: formic acid (v/v) was chosen as the optimal mobile phase to effectively separate paracetamol from the main constituents in saliva. Suspected interferants from the saliva matrix were retained within a distance of 5 mm from the sampling spot, while paracetamol was transported to 10-15 mm from the sampling spot after PC within a timeframe of <5 minutes. To seamlessly integrate PC and PS, a novel arrow-shaped paper strip with a triangular head and a rectangular stem was designed. The arrow design (in particular the stem) and sample application (position and volume) were also optimised. With the optimal mobile phase, paper shape and sample application position confirmed, the method of PA-MS to detect paracetamol in saliva was extensively investigated.

To verify the hypothesised main advantage of PA-MS, namely a reduction of the matrix effect, 2 μL of 100 μg/mL paracetamol spiked in raw saliva or water were applied onto a traditional triangular (PS) or arrow-shaped (PA) paper substrate for subsequent MS analysis, respectively. The intensities of [M+H]⁺, [M+Na]⁺, and [M+K]⁺ were compared. Briefly, when detected with traditional PS-MS, the intensity of [M+H]⁺ in saliva was suppressed to only 13.6% of the intensity in deionised water (P=0.0006). When detected with the novel method of PA-MS, the signal of [M+H]⁺ in saliva was about 93.4% of the intensity in water (P=0.7133). With more than 10 fold enhancement of the intensity of [M+H]⁺, PA-MS almost eliminated the matrix effect of saliva.

Perhaps more importantly, the coefficient of variation (CV) of individual saliva collected from 7 participants was reduced from 16.3% (PSI-MS) to 9.5% (PA-MS). These data indicate that PA-MS can effectively reduce the salivary matrix effect.

Following the method validation in the lab, a clinical cross-validation study by comparing PA-MS with the gold standard test used currently was done. Seventeen adult participants, averaging 36.8 years in age, 5 males and 12 females with 12 identifying as white and 5 as non-white, were recruited for the study. Blood, resting saliva (RS), and stimulated saliva (SS) were collected before and at 15, 30, 60, 120 and 240 minutes after the ingestion of 1.0 g acetaminophen. Participants experiences of anxiety, discomfort, and convenience during the collection of blood, RS and SS, as well as their preferences, were assessed using a self-designed questionnaire.

The results of blood samples, analysed using the current clinical test, served as the gold standard to validate our new approach. Specifically, the results of blood samples obtained with PA-MS were compared with the gold standard to validate PA-MS using the same biofluid. Furthermore, the results of saliva samples obtained with PA-MS were also compared with the gold standard to ascertain the reliability of RS and/or SS. Adhering to established guidelines, the validation was characterised using Lin's concordance correlation (denoted as pc), Bland-Altman difference plots, and ratios of PA-MS over the gold standard plotted at each sample collection time. Participants' perceptions and preferences regarding the three sample collection procedures were delineated by mean rank and compared by the Kruskal-Wallis test. Statistical significance was set at p<0.05.

### Results and Discussion

### 1. Design and Development of PA-MS/MS

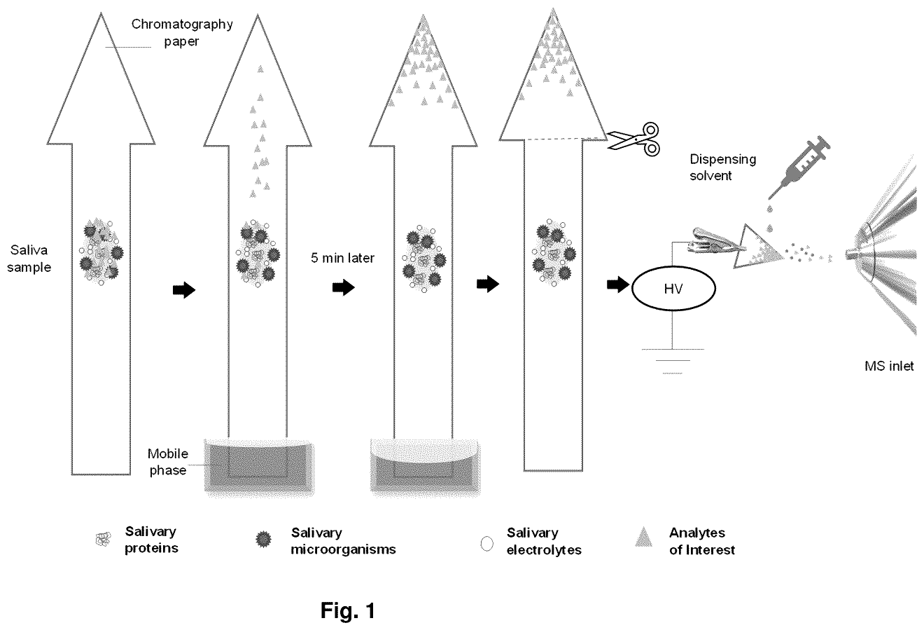

Paper chromatography (PC) is a long-established method for separating mixtures [35]. We hypothesized that it should be possible to effectively and seamlessly integrate PC and traditional PS in an efficient and effective manner. To do so, three different paper designs were required (FIG. S-1 in supporting information): serrated paper strips were used to optimize PC separation, to identify the location of analytes (FIG. S-1a), triangular (isosceles) paper was used for traditional PS-MS analysis (FIG. S-1b), and arrow-shaped paper strips were the final platform design used for PA-MS (FIG. 1). The basic concept is illustrated in FIG. 1, showing how the analyte of interest is effectively and sufficiently separated from the matrix, to enable an almost matrix-free PS analysis to be carried out. To ensure that PA-MS retained the characteristic benefits of PS (in particular, simplicity and speed) notwithstanding the additional benefits it affords, the process can be summarised as three simple steps (FIG. 1): (i) sample application, (ii) paper chromatography (dip the paper strip in to the mobile phase), and (iii) isolate the paper triangle (e.g., by cutting, for analysis by PS-MS).

### 1.1 PC Mobile Phase Selection

The development of PA-MS began by first selecting a suitable mobile phase, which is a key consideration for the success of PC [35]. For classic PC, it is required that the analyte(s) being separated can be visualized to optimize and perform an analysis. Thus, detection requires that the analyte(s) can be visible (e.g., analyte(s) naturally has a distinct color; treatment with a suitable reagent to enable analyte visualization; analyte(s) absorbs UV-radiation to leave a dark spot against the natural fluorescence of chromatography paper, etc.). To determine a suitable mobile phase that can extract and separate paracetamol from saliva efficiently, the process was optimized by using serrated paper strips. As shown in FIG. S2a (supporting information), 2 μL of 100 μg/mL paracetamol in saliva was applied onto the center of region 0 of the serrated paper strip. The end near to the origin (as denoted in FIG. S2a) was immersed by 5 mm into the mobile phase, and the solvent front travelled to the far end of the paper by capillary action. When the front of the mobile phase reached region 10 (i.e., the 10th sawtooth from the origin, as denoted in FIG. S2a), the paper strip was taken out of the flask and left to dry in the open air for 1 min. Then the serrated comprising regions 0-10 were manually cut and subject to PS-MS analysis in positive mode. Details regarding the MS conditions can be found in supporting information (Methods, Section 4.1).

With the literature as our guide, pure ethyl acetate was initially trialled as the mobile phase but failed to sufficiently transport paracetamol away from the origin; the protonated molecular ion peak of paracetamol, [M+H]⁺, was most intense in the region closest to the origin, even after PC (FIG. S2b). Upon further inspection intense sodium, [M+Na]⁺, and potassium, [M+K]⁺, adducts were also observed; the signal intensity of [M+Na]⁺ was similar to that of [M+H]⁺ at the origin when pure ethyl acetate was used as the mobile phase (FIG. S2c). In an attempt to suppress metal ion adduct formation, formic acid and volatile ammonium salts were utilized.

Thus, in a bid to enhance the signal intensity of the protonated molecular ion, [M+H]⁺, in saliva, 10% formic acid and two concentrations of NH₄HCO₂ in ethyl acetate were tried as the mobile phase. The intensity of each serrated region after PC was compared with that of 2 μL 100 μg/mL of paracetamol in water or saliva with classic PS-MS/MS. As the results in FIG. S2d show, PC with 50 mM NH₄HCO₂ in 9:1 ethyl acetate: formic acid (v/v) as the mobile phase could produce the highest intensity of [M+H]⁺ at a distance (15 mm) that was deemed sufficiently far enough from the origin (region 3, FIG. S2d). Thus, the mobile phase used for PA-MS analysis in this study was confirmed to be 50 mM NH₄HCO₂ in 9:1 ethyl acetate: formic acid (v/v).

### 1.2 Locating Saliva Constituents After PC

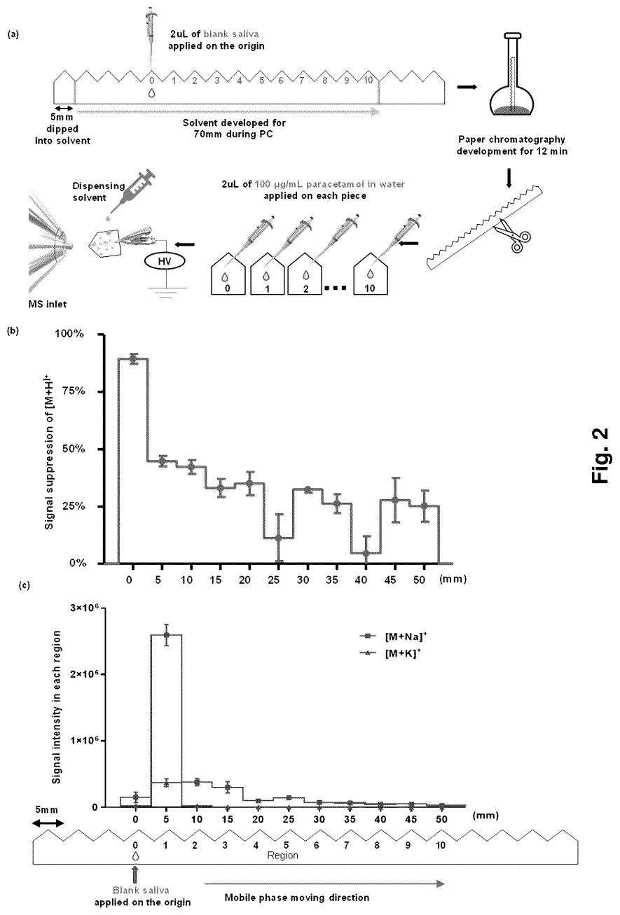

After confirmation of mobile phase, the second step was to determine the distance travelled by salivary components during PC, to ensure a sufficient separation from the analyte of interest (paracetamol). As a biofluid, human saliva contains an enormous amount of organic and inorganic components, as well as microorganisms.[39] For instance, it is estimated that there are about 2.2 mg/ml of proteins in saliva,[39] which includes as many as 3449 different proteins.[40] The concentrations of sodium and potassium electrolytes in saliva are in the region of 0.15 to 0.60 mg/ml and 0.8 mg/ml, respectively.[39] Besides viruses and fungi, over 770 prokaryotic species have been identified in saliva.[41] The complicated constitution of saliva means it is impossible to find out the distance travelled by each specific component. Thus, the extent of ion suppression was determined by considering the signal intensity of the protonated molecular ion, [M+H]⁺, as an indicator of the position of salivary interfering components. [M+Na]⁺ and [M+K]⁺ were used to estimate the positions of salivary sodium and potassium after PC. The experimental process is illustrated in FIG. 2(a). Briefly, 2 μL blank human saliva was applied and 12 min of PC was done with 50 mM NH4HCO2 in 9:1 ethyl acetate: formic acid (v/v). Then, regions 0-10 were cut in to separate paper spray type substrates, and 2 μL 100 μg/mL paracetamol in deionised water was applied onto each piece of paper. After being allowed to dry in the open air, PS-MS was conducted on each substrate (as described in supporting information). As shown in FIG. 2(b), the intensity of [M+H]⁺ at the origin (region 0) was reduced to about 20% of the normal 1×10⁸ signal intensity of [M+H]⁺ in water. This result indicates that most salivary components that suppress the signal of [M+H]⁺ were not transported by the mobile phase during PC development. This trend can also be observed by examining full scan mass spectra representative of each region whereby the protonated molecular ion peak gradually becomes the more intense in successive regions (FIG. S3). These results tend to imply that most salivary components are not effectively transported during PC, thus offering a promising foundation for extraction and separation of paracetamol from saliva.

### 1.3 Ensuring Sufficient Analyte Separation

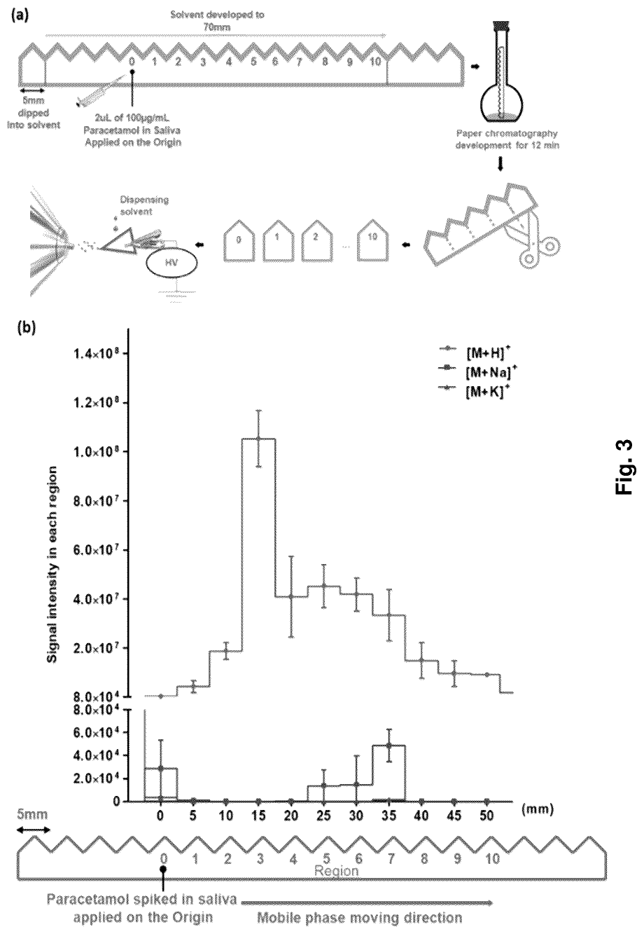

Since it was inferred that most of the saliva constituents remained close to the origin, the third step was to identify the location where most of the paracetamol was transported after PC. In contrast to the procedure for step 2 (noted in the prior text), in this experiment 2 μL of 100 μg/mL paracetamol in saliva was applied at the origin and no more sample was added after PC (FIG. 3(a)). The intensities of [M+H]⁺, [M+Na]⁺, and [M+K]⁺ of each piece of paper are plotted in FIG. 3(b). The intensity of [M+H]⁺ at the origin was as low as 1×10⁵, while the peak of [M+H]⁺ in the 3rd region (15 mm from the origin) was as high as 1×10⁷, ˜200 times the signal intensity observed at the origin. The differences were statistically significant with a P value lower than 0.001. These data indicate that most of the paracetamol is transported away from the origin by the mobile phase, which makes it possible to be sufficiently separated from the saliva matrix. Interestingly, the peak intensity of [M+Na]⁺ was reduced from 2.59×10⁶ to 4.85×10⁴, and [M+K]+ almost disappeared comparing FIG. 2(b) with FIG. 3(b). The significant drop off in intensity of these metal ion adducts at the origin is because most of the paracetamol has been transported away from this region. These data show that metal ion adducts of paracetamol can be successfully decoupled by the process of PC. Thus, at this juncture, it can be concluded that paracetamol can be effectively extracted and separated from saliva by PC with 50 mM NH₄HCO₂ in 9:1 ethyl acetate: formic acid (v/v).

### 1.4 PA Substrate Design: Seamless Integration of PC and PS

Following the successful separation by PC, the fourth step was to explore how best to integrate PC and PS in to a seamless and efficient workflow. Prior efforts in the literature to combine PC and PS were ad-hoc and essentially relied on visual detection to identify regions that could be cut in to triangles for subsequent PS-MS analysis.[29] [42] [43] The basic methodology used in the prior attempts is illustrated in FIG. S4 (supporting information). A major drawback of this approach is that analyte diffusion during PC is not effectively controlled, which can lead to a significant loss of analyte signal intensity. It is well known that chemicals unavoidably diffuse in the mobile phase during chromatography [44]. The diffusion of paracetamol is also evident in our optimization results using serrated paper strips. For instance, in FIG. 3(b), the protonated molecular ion, [M+H]+, can be detected with an appreciable signal intensity in region 2 and even as far as region 10.

Aiming to solve the problem caused by diffusion, an arrow-shaped paper was designed and the

PC process was modified based on the insight gleaned from the prior investigations. The dimensions of the arrow shaped paper can be found in FIG. S1 (supporting information). The modified process of PC is illustrated in FIG. 4a. The length of the paper was chosen such that mobile phase front would reach the tip of arrow within 5 minutes, to allow the paracetamol molecules to be transported to the tip of the triangular-arrow head. Furthermore, three positions of sample application were considered and compared in relation to signal intensity. The result in FIG. 4b shows that the signal intensity of [M+H]+ reached 1×10⁸ with only 2 μL of 100 μg/mL paracetamol, and the sample applied at the 10 mm position produced the highest average intensity of 2.37×10⁸. One-way ANOVA and Tukey's multiple comparison test showed the differences were statistically significant (P=0.0032).

The separation effect of PA-MS was visualised in FIG. 3(c), in which arrow-shaped paper strips were dyed with spray of 2.5 mMFeCl₃, 0.5 mM K₃Fe(CN)₆ and 0.5M HCl in deionised water before or after PC.[45] The chemical reaction behind the dyeing process were listed as Equation 1 and Equation 2 (Supporting Information). Components in saliva that were slightly dyed by Prussian blue (KFeIII [FeII (CN)₆]) still stayed at the origin of sample application after chromatography, while paracetamol was transported to the tip of the arrow head by the mobile phase.

Thus, the PA-MS method taken forward for salivary paracetamol analysis consisted of: 2 μL of sample to be applied on the arrow-shaped substrate at a distance of 10 mm from the base of the arrow head (as indicated in FIG. 4(a)). PC with 50 mM NH₄HCO₂ in 9:1 ethyl acetate: formic acid (v/v) mobile phase is applied for 5 minutes, after which the arrow head is cut off and subjected to PS-MS type analysis.

### 1.5 PA-MS Verification

Now, the last question to answer is whether the PA-MS approach designed and developed in this study could significantly improve upon the performance of traditional PS-MS. 2 μL 100 μg/mL paracetamol in saliva or water were applied onto a triangle paper or arrow-shaped paper for PS-MS and PA-MS analysis, respectively. Each experiment was performed in triplicate. The intensities of [M+H]⁺, [M+Na]⁺, and [M+K]⁺ were compared. Details of the experiment can be found in the supporting information (Section 4). The grade of ion suppression (matrix effect), is determined by the matrix factor (MF), which is defined by the following equation: MF %=(Peak response in presence of matrix/Peak response in solvent)×100. When there is no matrix effect (i.e., no ion suppression), MF should be equal 100% [46]. Variation of MF more than ±15% indicates the presence of what might be deemed as unacceptably high matrix effects.

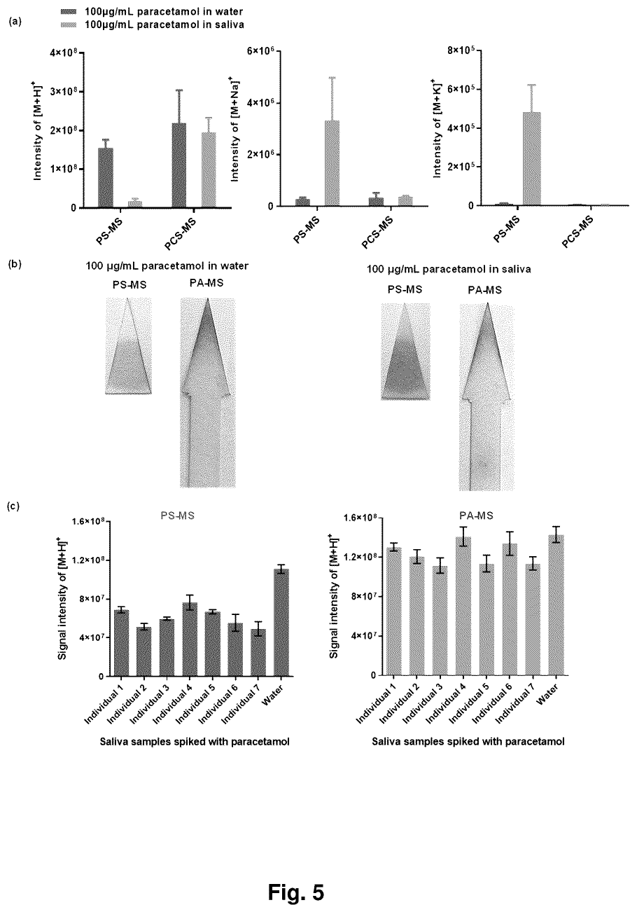

The results in FIG. 5 fully demonstrated the advantages of PA-MS. When detecting with traditional PS-MS, the intensity of [M+H]⁺ in saliva was suppressed to 1.70×10⁷, which was only 13.6% of the intensity in deionised water (P=0.0006). When detecting with the novel method of PA-MS, the signal of [M+H]⁺ in saliva was 1.96×10⁸, about 93.4% of the intensity in water (P=0.7133). With more than 10 fold enhancement of the intensity of [M+H]⁺, PA-MS almost eliminated the matrix effect of saliva. Results of [M+Na]⁺ and [M+K]⁺ also supported the conclusion (FIG. 5a). Furthermore, even the signal of [M+H]⁺ in water samples was improved from 1.56×10⁸ in PS-MS to 2.20×10⁸ in PA-MS (though P=0.2691). The improvement of signal in water implied that PA-MS may concentrate the analytes to some extent. The hypothesis was supported by the visualized distribution of paracetamol in FIG. 5(b). Obviously, paracetamol was concentrated to the tip of arrow-shaped paper after PC. In FIG. S5 of the SIM chromatograms of PA-MS, [M+H]⁺ reached peak height immediately when the voltage applied onto the paper, while the intensity of [M+H]⁺ during PS-MS was gradually increased. This result indicated that the modified PC that let mobile phase flow to the end of paper concentrated paracetamol near the apex.

Considering saliva's composition varies significantly with an inter-individual variability as high as 57%, 34 the matrix factors among individuals was supposed to vary accordingly when detecting paracetamol by PS-MS. With separation of paracetamol from the saliva matrix, PA-MS may help to control the inter-individual variability. To test this hypothesis, 7 participants were recruited and their resting saliva was collected. Demographic information of 7 participants was summarised in supporting information (Table S1). The 7 participants' saliva samples were spiked with the same concentration of 100 μg/mL paracetamol, and then detected by PA-MS and PS-MS. As seen in FIG. 5(c) and Table 1, for PS-MS, the average matrix factor was 64.0%, and the coefficient of variation among 7 participants was 16.9%, not reaching the requirement of ±15%³⁵. While the matrix factor of PA-MS was improved to 87.9% and the CV was controlled to 9.5%. These results further confirmed that PA-MS could sufficiently control the matrix effect of saliva.

So far, the method of PA-MS for salivary paracetamol detection was developed and its aim was fulfilled. With a simple, rapid and easy process of PC, the intensity of [M+H]⁺ was improved by more than 10 times, and the matrix factor and CV of inter-individuals were corrected into the required range of ±15%.

TABLE 1
Intensity of [M + H]+ of 2 μL 100 μg/mL Paracetamol
in 7 Participants Detected by PS-MS and PA-MS

| Detection | Mean of | SD of | CV among | Matrix |
|---|---|---|---|---|
| Method | Individuals | Individuals | Individuals^{[a]} | factor^{[b]} |
| PS-MS | 6.11E+07 | 9.94E+06 | 16.3% | 64.0% |
| PA-MS | 1.26E+08 | 1.19E+07 | 9.5% | 87.9% |

^{[a]}CV among individuals = SD of 7 participants / Mean of 7 participants.
^{[b]}Matrix factors = Mean of 7 participants / Mean of water.

### 2 Method Validation of PA-MS/MS

### 2.1 LOD, LOQ and Linearity of Calibration Curve

To validate the method of PA-MS/MS, paracetamol-D4 was used as internal standard (IS). Different concentrations of paracetamol and 500 ng/ml paracetamol-D4 were spiked with deionised water or human resting saliva. Five sets of experiments were conducted: paracetamol in raw saliva detected by PA-MS/MS, paracetamol in raw saliva detected by PS-MS/MS, paracetamol in pure water detected by PA-MS/MS, paracetamol in pre-treated saliva detected by PS-MS/MS, and paracetamol in pre-treated saliva detected by ultra-performance liquid chromatography (UPLC)-MS. Each set was replicated 3 times. PA-MS and traditional PS-MS/MS were conducted on a Thermo Scientific Orbitrap Exploris 240 mass spectrometer (Thermo Fisher, Waltham, MA, USA). The Selective Reaction Monitoring (SRM) transitions were: m/z 152.0706→110.0600 (quantifier) and m/z 152.0706→65.071 (qualifier) for paracetamol, and m/z 156.0957→114.0850 (quantifier) and m/z 156.0957→69.090 (qualifier) for paracetamol-D4. UPLC-MS/MS was conducted on a Waters ACQUITY UPLC system (Waters Corporation, Milford, MA). The multiple reaction monitoring (MRM) transitions monitored were: m/z 151.94→109.95 as a quantifier and m/z 151.94→92.84 as a qualifier for paracetamol and m/z 155.96→114.05 as quantifier and m/z 155.96→96.69 as qualifier for paracetamol-D4. The other parameters of the experiments are in the supporting information (Section 3).

The average peak area ratios of quantifier of paracetamol over that of paracetamol-D4 were plotted to the theoretical concentrations. Each constructed calibration curve consisted of 7 concentration levels, ranging between 5-1000 ng/ml for detection with PA-MS/MS and 90-4500 ng/ml for detection with UPLC-MS. The linear regression equations and the squares of correlation coefficients (r²) were noted in FIG. S6. The limits of detection (LODs) and the limits of quantitation (LOQs) of the 4 sets of experiments were listed in Table 1. More data were described in supporting information (Table S1 to S4).

The linearity of detection for paracetamol in saliva by PA-MS/MS reached the same level of UPLC-MS/MS. The LOD and LOQ for paracetamol in saliva by PA-MS/MS were 61.10 ng/ml and 185.14 ng/ml, respectively. Both were very close to the LOD and LOQ of detection in water by PA-MS/MS. Furthermore, they were even lower than the LOD and LOQ of detection by UPLC-MS. These results indicated that PA-MS/MS could treat saliva samples efficiently.

Comparing with other studies of direct detection of paracetamol in biofluids, the sensitivity achieved with PA-MS/MS was much better than an LOQ of 2.9 μg/mL reported in 2020.[46] In that study, the serum sample was 5 times diluted, and the novel electrokinetic extract syringe only helped to remove proteins.[46] In comparison, for PA-MS/MS, the raw samples were applied directedly; no complicated apparatuses were added; deproteination and desalting were fulfilled simultaneously by the process of PC; and more importantly, the analytes were concentrated rather than diluted. These features contributed to the enhanced performance of PA-MS/MS and potentially make it easier to be introduced into practice.

However, lower LODs and LOQs of paracetamol detection were reported in studies that treated biofluid samples with traditional methods like protein precipitation before detection. For example, Richard Kin-ting Kam et al. reported an LOQ of 125 ng/ml with 20 μL blood serum sample of paracetamol by LC-MS. However, they defined LOQ as S/N ratio higher than 10.[47] If taking the same criterion for PA-MS, the LOQ of this study would be 5 ng/ml with a S/N of 135.93 (Table S1). Also, some studies used ±15% of CV and accuracy as the criterium of LOQ, and reported a LOQ of 20 ng/mL with 50 μL of sample [48]. PA-MS/MS' results showed that the CV and accuracy of 50 ng/ml paracetamol in saliva were 3.4% and 3.2%, respectively with only 2 μL of saliva sample (Table S1). More importantly, complicated and time-consuming sample pretreatments were conducted in those studies. In summary, with a smaller volume of sample and simpler treatment procedure, PA-MS/MS's sensitivity is at comparable to other methods that used traditional sample treatments.

Recently, a novel nitrogen-doped carbon@TiO₂ double-shelled hollow sphere based electrochemical sensor was developed for determination of paracetamol in human serum and saliva. The LOQ of this biosensor was reported as 300 ng/ml [49]. This means the sensitivity of this simple method of PA-MS/MS is also comparable to other non-standard techniques.

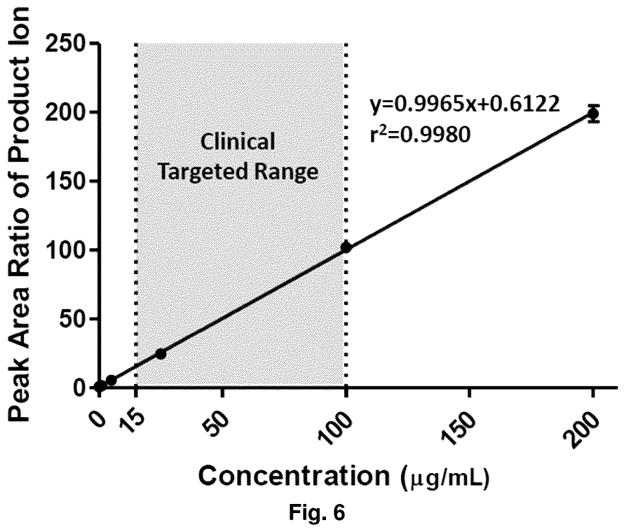

In clinical practice, paracetamol is usually detected in overdose patients with higher concentration [53]. Currently, in the UK, the treatment line of paracetamol concentration is 100 μg/mL at 4 hours after ingestion and 15 μg/mL at 15 hours after ingestion [54]. Thus, a calibration curve of 0.2-200 μg/mL was constructed (FIG. 6). The accuracy and precision of 6 concentrations were listed in Table S7.

TABLE 2
LODs and LOQs of paracetamol detection in saliva
and water by PS-MS/MS, PA-MS/MS and UPLC-MS/MS.

| | | | | SD of | LOD^{[a]} | LOQ^{[b]} |
|---|---|---|---|---|---|---|
| Matrix | Method | r² | Slope | Response | (ng/mL) | (ng/mL) |
| Raw Saliva | PA-MS/MS | 0.9969 | 0.002 | 0.03 | 61.10 | 185.15 |
| Pure Water | PA-MS/MS | 0.9971 | 0.002 | 0.04 | 60.96 | 184.71 |
| Raw Saliva | PS-MS/MS | 0.9864 | 0.002 | 0.01 | 135.91 | 411.83 |
| Pre-treated | PS-MS/MS | 0.9941 | 0.002 | 0.04 | 93.38 | 282.98 |
| Saliva^{[c]} | | | | | | |
| Pre-treated | UPLC- | 0.9967 | 0.001 | 0.02 | 65.18 | 197.50 |
| Saliva^{[c]} | MS/MS | | | | | |

^{[a]}LOD = 3.3 * SD of Y-Intercept/Slope.
^{[b]}LOQ = 10 * SD of Y-Intercept/Slope.
^{[c]}Treatment of sample: samples were spiked with methanol (1:4, v/v), cooled for 30 min under −20° C., and then centrifuged for 20 min with 14000 rpm under 4° C. The supernatant was diluted 4 times with deionised water.

### 2.2 Extraction Recovery and Matrix Factors

Basically, the main feature of PA-MS/MS was to pre-treat samples with the process of PC. Thus, extraction recovery and matrix factors were essential figures of merit by which to evaluate PA-MS/MS. FIG. S7 in supporting information showed how the experiment of extraction recovery was conducted. Details of relevant experiments can be found in supporting information. The calculations of extraction recovery and matrix factor were described in Equations 3-6 in supporting information. According to the guidelines published by U.S. Food and Drug Administration (FDA) and European Medicine Agency (EMA), the criteria of recovery and matrix factors are defined as 100+15%.[55] As seen in Table 2, acceptable recovery was attained at all concentration levels of paracetamol.

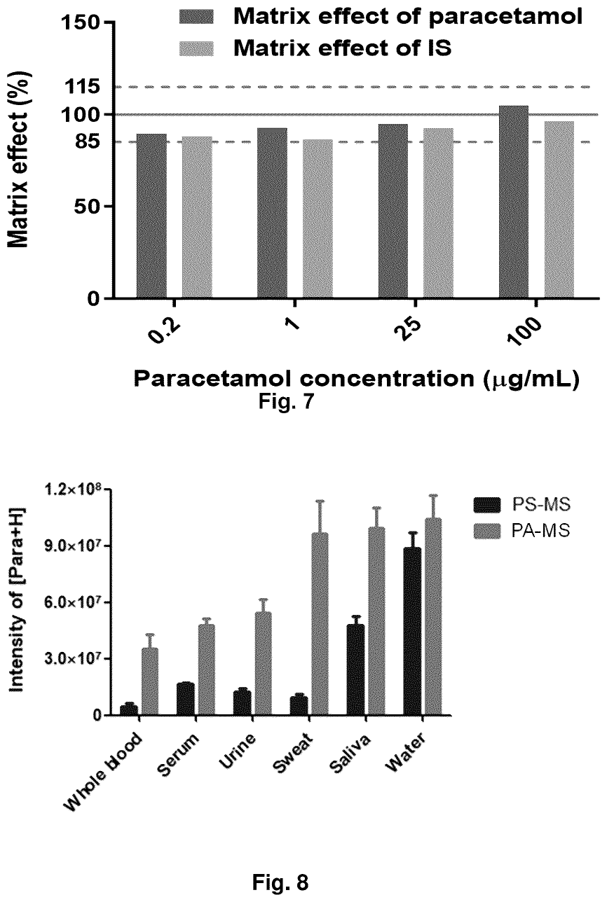

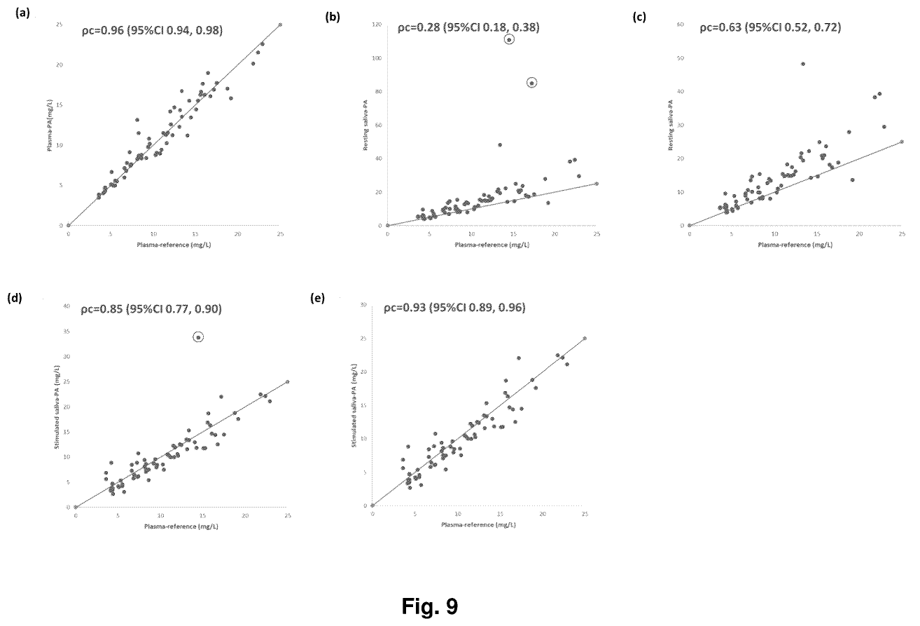

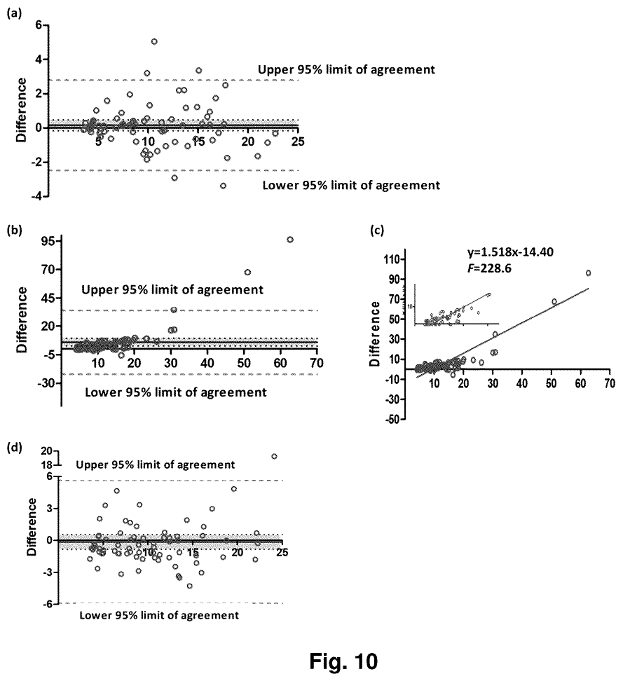

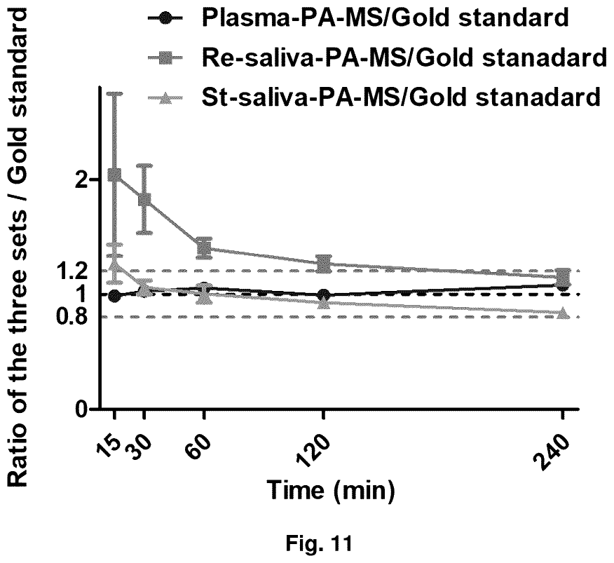

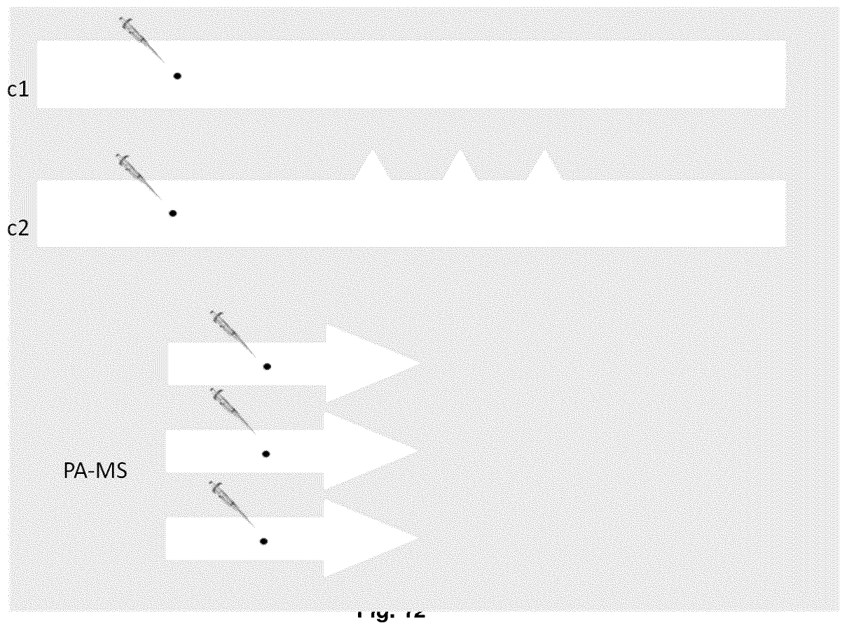

FIG. 7 shows the matrix factors of PA-MS/MS at 4 concentration levels. Matrix factors of of paracetamol and Paracetamol-D4 were in the range 89.5-105.0% and 88.1-96.5%, respectively. The internal standard (IS) normalised matrix factors were 101.6-108.8%.

TABLE 3
Extraction Recovery of Paracetamol
Detection in Saliva by PA-MS/MS.

| Concentration | | Peak Area Ratios | | |
|---|---|---|---|---|
| (μg/mL) | Spiked time | Mean | SD | Recovery^{[a]} |
| 0.2 | before-chroma | 0.274 | 0.026 | 103.3% |
| | Post-chroma | 0.265 | 0.043 | |
| 1.0 | before-chroma | 0.929 | 0.016 | 104.9% |
| | Post-chroma | 0.886 | 0.023 | |
| 25 | before-chroma | 20.333 | 0.491 | 99.5% |
| | Post-chroma | 20.425 | 0.823 | |
| 100 | before-chroma | 84.601 | 1.539 | 101.6% |
| | Post-chroma | 83.246 | 1.624 | |

^{[a]}Recovery = Peak area ratio of before-chroma spiked samples / Peak area ratio post-chroma spiked samples.

### 2.3 Intra-Day and Inter-Day Precision

The method accuracy and precision were evaluated for PA-MS/MS with 4 concentration levels on three days and replicated for 3 times on each day. To sum up, the precision of intra-day and inter-day all satisfied the requirements of FDA and EMA.[50] More details can be found in supporting information (Table S8).

### Alternative Biomedia

In addition to salivary analysis of paracetamol, additional biomedia (also known as matrices) were examined to determine the wider applicability of the PA method developed herein. Briefly, 100 μg/mL paracetamol was spiked with horse whole blood, bovine serum, and human urine, and then 2 μL of samples were applied onto the arrow-shape paper for PA-MS/MS, or normal triangle paper for PS-MS/MS (FIG. 8).

### 3 Clinical Validation of PA-MS/MS

### 3.1 Paracetamol in Plasma Can be Reliably Measured by PA-MS

As shown in the results on Fig, concentrations of paracetamol in plasma measured by PA-MS demonstrate a high agreement with that detected by Abbott enzyme-based assay, the gold standard test. All criteria of cross-validation were met, indicating that PA-MS is a reliable method to detect paracetamol.

Mass spectrometry had been used to measure paracetamol in bio-samples like plasma and urine since 1970s.³⁶ Although this method is common to conduct in research laboratories, mass spectrometry is not widely used in clinical practice to detect paracetamol. The main reason is that bio-samples have to be pre-treated by liquid-liquid extraction, solid phase extraction, protein precipitation, or dilution with organic solvents before conducting liquid or gas chromatography.³⁷⁻⁴² In our previous study to detect paracetamol in saliva by LC-MS, sample preparation process took more than one hour.¹⁵ That is why so far no hybrid mass spectrometry is taken as a routine test to measure paracetamol in clinic.

Comparatively, the novel PA-MS we developed combined sample collection, extraction, enrichment, separation, and ionization onto a single piece of paper, the entire process, from sample to result, can be completed in under 10 minutes, while enhancing performance. The approach achieved a LOQ for plasma paracetamol, as low as 0.21 μg/mL, with excellent linearity across the range of 0.2-200 μg/mL, using only 2 μL of sample volume.

To sum up, PA-MS successfully passed the cross-validation with the gold standard test. With its advantages over traditional mass spectrometry, PA-MS offers a reliable and convenient way to detect paracetamol in plasma.

### 3.2 Paracetamol Concentrations in Resting Saliva Were Higher Than in Stimulated Saliva

It is well known that resting saliva is different from stimulated saliva in terms of secreting glands, pH value, flow rate, and composition.^{43, 44} These differences have been proved influencing the excretion of endogenous and exogenous salivary components, like immunoglobins, hormones and drugs.⁴⁵⁻⁴⁸ However, as we know by best, no one has compared paracetamol's concentration in resting saliva with stimulated saliva so far. Aiming to fill this knowledge gap, we measured paracetamol in the two kinds of saliva by PA-MS, and for the first time, the differences between them were identified.

Table 6 and FIG. 7 demonstrated that concentrations of paracetamol in resting saliva were always higher than stimulated saliva by about one third. Paracetamol's concentrations in resting saliva were much higher than in plasma at 15 min after ingestion of paracetamol, and the differences between them became less and less as the time passed. Similarly, paracetamol concentration in stimulated saliva was higher than in plasma at beginning, but the difference between them was reversed after 120 min.

With high permeability and high fraction unbound to plasma proteins, paracetamol can be easily and rapidly transferred from blood into saliva along concentration gradient.^{49, 50} However, this cannot help to explain why salivary paracetamol is higher than plasma. Here we put forward a hypothesis to explain the higher paracetamol concentration in saliva. Primary saliva is secreted from the acini of saliva glands. When the initial saliva flows through the ducts of saliva glands, NaCl, water and other electrolytes are reabsorbed by duct cells. Supposing paracetamol cannot be reabsorbed,⁵¹ it will be concentrated somewhat after travelling through the ductal system. This can explain why salivary paracetamol is higher than blood. Furthermore, resting saliva has a slower flow rate than stimulated saliva,⁵² so paracetamol in resting saliva can be concentrated more than in stimulated saliva, which is consist with our observation (Table 6 and FIG. 7). However, this hypothesis cannot explain why the differences between saliva and plasma become less and less as the time passes. More studies deserve to be dedicated into exploration of mechanism behind this phenomenon.

### 3.3 Resting Saliva Measured by PA-MS Showed Poor Agreement With Gold Standard While Stimulated Saliva Provided a Better Agreement

As to the agreement with gold standard, resting saliva detected by PA-MS showed poor correlation with the gold standard method, with a pc of 0.63 even after excluding two abnormal values (FIG. 5c), much lower than the pre-setting criterium of 0.85. Blank-Altman plot indicated a systematic error between resting saliva detected by PA-MS and gold standard. As demonstrated in FIG. 6c, a positive correlation existed between the differences and means of resting saliva detected by PA-MS and gold standard, suggesting that the larger the mean of the two measurements, the larger the difference. Furthermore, ratios of Resting saliva-PA-MS/Gold standard were between 1.15-2.04 (mean: 1.53), and only 22 out 70 pairs (31.4%) were within 1.0±0.2. With failure to reach the relevant criteria of cross-validation, resting saliva measured by PA-MS showed a poor agreement with gold standard.

Different from resting saliva, the pc of stimulated saliva detected by PA-MS with the gold standard method was 0.93 when excluding that abnormal value (FIG. 5e). The mean of differences between stimulated saliva detected by PA-MS and gold standard was −0.14 mg/L (95% CI: −0.84, 0.56), including 0. Ratios of stimulated saliva-PA-MS/Gold standard were between 0.84-1.27 (mean: 1.02), and 47 out of 70 pairs (67.1%) were within 100±20%. These results fulfilled most requirements of cross-validation and implied that stimulated saliva detected by PA-MS has a better agreement with gold standard than resting saliva, indicating that stimulated saliva would be a preferable biofluid over resting saliva to replace blood for paracetamol detection.

As to the best of our knowledge, the earliest paper explored salivary paracetamol concentration was published in 1973, but saliva collection method was not mentioned in it.⁵³ Several other studies did not report saliva collection methods too, which highlighted the ignorance of the importance of standard saliva collection procedures.⁵⁴⁻⁵⁶ Surprisingly, some researchers even wrongly reported chewing stimulated saliva as resting saliva.⁵⁷ Considering the differences between paracetamol's concentrations in resting saliva and stimulated saliva we found in this study, the previous studies' results may be challenged without clarifying sample collection method.

In summary, this study's findings affirm the need to establish and standardise collection methods before salivary paracetamol is used for diagnostic purposes. In terms of paracetamol detection, stimulated saliva would be preferable over resting saliva. More importantly, it is reasonable to deduce that when analysing any molecules in saliva, comparison between resting saliva and stimulated saliva should be conducted without exemption.

### 3.4 Stimulated Saliva Were Preferable Mostly Because of Least Anxiety and Discomfort Caused by Sample Collection

User-centred design (UCD) is a design approach that prioritizes the needs and preferences of users in the development process of products or systems. In the medical device development, UCD is crucial as medical devices directly affect the health and well-being of patients.⁵⁸ So far, few studies have explored personal experiences of sample collection processes.⁵⁹ By adopting UCD principles, this study investigated participants' feelings and preferences of the collection processes of blood, resting saliva and stimulated saliva by a questionnaire.

Blood sample is collected invasively through a cannula or needle inserted into the veins. Under some difficult conditions, 10 out of 18 participants were tried for 3-11 times before a cannula was successfully inserted into the veins. So, no doubt, blood sample collection procedure was listed as the one caused most anxiety, most discomfort, least convenience, and it was ranked the least preferable method by participants (Table 7). These results highlighted the need to develop non-invasive ways to substitute blood samples.

In this study, stimulated saliva was collected by chewing the cotton swabs. It brought least anxiety and most convenience, and it was preferred mostly by the participants (Table 7). However, discomfort caused by stimulated saliva was higher than resting saliva. In the open-ended questions of the questionnaire, some participants feedbacked the following comments about stimulated saliva collection:
“Chewing cotton is not pleasant at all.”
“The cotton swab has a strong unpleasant taste.”
“The dry cotton in the mouth is not pleasant at all, the dryness needs to solved.”
“Chewing the cotton swab is more unpleasant than a 22 G needle.”

These comments explained the discomfort caused by stimulated saliva and pointed out the drawbacks of the method we used to stimulate saliva.

### 3.5 Saliva Measured by PA-MS has Practical Advantages Over Gold Standard Test

To fully compare the features of the three testing sets with the gold standard test, sample collection methods were summarised in Table 8, and samples detection methods were compared in Table 9. Clearly, saliva detected by PA-MS took less time, consumed less supplies and human costs, which make it more competitive than the current gold standard test.

TABLE 8
Comparison of sample collection

| Concerns | Saliva | Blood |
|---|---|---|
| Volume of sample for per | 2 μL | 1.3 mL |
| test | | |
| Supplies to collect sample | saliva collection | blood collection tube, tourniquet, disinfection wipe, |
| | tube | needle/cannula, medical adhesive, syringe, gauze, |
| | | plaster |
| Sample collection time | 2 min/1 min ^{a} | 5-15 min (Depends on the difficulty of blood collection) |
| Sample collection failure | No | 10 out of 18 failed for 3-11 times |
| Discomfort | Less | More (pain, bruise, anxiety, faint) |
| Professional staff | No | Band 5 + nurse |
| Cost of sample collection | £1-2/sample | £10-30/sample |

^{a} In this study, resting saliva was collected in 2 minutes and stimulated saliva was collected in 1 minute.

TABLE 9
Comparison of sample detection

| | | | Gold standard | |
|---|---|---|---|---|
| Concerns | PA-MS/MS | | test | |
| Volume of sample for per test | 2 | μL | 50 | μL |
| Sample preparation time | 5 | min | 5 | min |
| Volume of solvent for sample preparation | 25 | μL | — | |
| Detection time | 1.6 | min | 16 | min |
| Volume of solvent for detection | 40 | μL | 150 | μL |

### Summary of Process of Method Development

### Spray Solvent Optimisation

Aiming to suppress metal ion adducts of paracetamol, e.g. [M+Na]⁺ and [M+K]⁺, spray solvent was optimised from 0.5% Formic Acid in 1:1 Methanol:Water to 0.5% Formic Acid+10 mM Ammonium Formate in 9:1 Methanol:Water Methanol:Water.

### Mobile Phase Optimisation

Mobile phase optimisation was performed according to the fourth aspect which provides a method of selecting a mobile phase, for example for the method according to the first aspect, the method comprising:
providing a sample comprising a matrix, on a substrate, for example a monolithic substrate, comprising a stationary phase;
separating the matrix on the substrate in a direction using a first mobile phase;
dividing the substrate transverse, preferably orthogonally, to the direction;
providing analytes on the substrate, for example on each division of the divided substrate after dividing and/or before dividing; and
analysing the analytes using mass spectrometry, wherein the substrate provides, at least in part, an ion source for ionising the analytes, for example wherein each division of the divided substrate provides, at least in part, a respective ion source for ionising the analytes.

At the first stage, pure ethyl acetate, methanol, and chloroform was used as mobile phase to extract paracetamol out of saliva matrix, ethyl acetate showed potential to be used.

The optimisation indicated that ammonium formate and formic acid may be helpful, so different concentrations of ammonium formate and formic acid was doped into ethyl acetate. After several experiments, 50 mM ammonium formate in 9:1 ethyl acetate:formic acid (v/v) was chosen.

### Locating Salivary Constituents After Paper Chromatography

As shown in FIGS. 2(a), 2(b) and 2(c), the suppression of paracetamol and the formation of metal ion adducts were only seen around the origin of blank saliva sampling spots. These results supported that the interferent salivary components were not effectively transported by the mobile phase. Also, the colorimetry pictures in FIG. 4(c) was consistent with the results of FIGS. 2(b) and 2(c).

### Locating Paracetamol After Paper Chromatography

Locating paracetamol after paper chromatography was performed according to the fifth aspect which provides a method of identifying a separation distance between analytes and a matrix, for example for the method according to the first aspect, the method comprising:
providing a sample comprising the analytes and the matrix, on a substrate, for example a monolithic substrate, comprising a stationary phase;
mutually separating the analytes and the matrix, on the substrate in a direction using a mobile phase;
dividing the substrate transverse, preferably orthogonally, to the direction; and
analysing the separated analytes using mass spectrometry, wherein the substrate provides, at least in part, an ion source for ionising the separated analytes, for example wherein each division of the divided substrate provides, at least in part, a respective ion source for ionising the separated analytes.

As shown in FIGS. 3(a) and 3(b), paracetamol was sufficiently transported by the mobile phase. In most cases, the peak intensity appeared around 15 mm from the sampling spots. Two phenomena in this section were noticed:
the position of the highest peak intensity varied between 15-40 mm in repeated experiments
paracetamol was diffused among the process of PC as seen in FIG. s2d, and FIG. 3(a).

### PA Substrate Design

The shape and size of triangle head was chosen as the traditional paper spray in order to compare the result of PA-MS with PS-MS.

The stem length of arrow-shaped paper was chosen based on the comparison of three distances of 5 mm, 10 mm and 15 mm from the bottom of the triangle as shown in FIGS. 4(a), 4(b) and 4(c).

This arrow-shaped paper and the seamless integration of PC to MS helped to overcome the influence of the variation and the diffusion of PC process mentioned in 4.4. This is a distinct feature that is different from the prior reported methods in the literature. In reports of other researchers, the variation of position and diffusion of analytes would keep being a problem.

### Summary of Experimental Protocol of PA-MS

Paper substrate: Whatman 1 CHR Chromatography paper, size: 250*250 mm

Different shapes of paper were cut by laser cutter and washed by methanol 5 min-water 5 min-methanol—in an ultrasonic bath.

Sample application: 2 μl of saliva spiked with paracetamol and paracetamol-D4 (internal standard) was added to the sampling spots, and dried up in less than 2 min.

5 min of paper chromatography: the stem end of arrow-shaped paper was dipped into the mobile phase by 2.5 mm, the mobile phase (50 mM ammonium formate in 9:1 ethyl acetate: formic acid) would reach the tip of triangle in less than 4 min, and the process of PC was prolonged 1 more minute to ensure paracetamol was concentrated near the apex of the triangle. After then, the arrow-shaped paper was allowed to dry for approx. 2 min.

Paper Spray process: the triangular head of the arrow-shaped paper was cut off. High voltage of +3500V was applied onto the bottom of triangle paper after 40 μl of spray solvent (0.5% formic acid +10 mM ammonium formate in 9:1 methanol:water) was added. The analysis was done with an orbitrap mass spectrometer (Exploris 240, Thermo) using Selected Reaction Monitoring (SRM) mode. Peak area ratio of product ion of paracetamol over product ion of paracetamol-D4 was used to quantify the concentration of paracetamol in saliva.

### Conclusions

The method of PA-MS effectively integrates PC with PS-MS yielding a significantly improved analytical performance, whilst retaining the signature benefits associated with classic PS. The process of PA-MS comprises 3 simple steps: direct sample application, paper chromatography and mass spectrometry analysis. Combining sample collection, extraction, enrichment, separation and ionisation onto the same piece of paper, the whole process from sample to result takes no longer than 10 min and is very easy to accomplish. With a sample volume of only 2 μL, the LOQ of salivary paracetamol detection reached as low as 0.018 μg/mL with excellent linearity over the range of 0.02-200 μg/mL.

With clinical validation study, PA-MS was further proved to be a reliable method to detect paracetamol in plasma. Resting saliva is excluded as a reliable biofluid to detect paracetamol. Stimulated saliva detected by PA-MS is a non-invasive, reliable, lower cost, more sensitive and convenient method for paracetamol detection. It is a promising candidate of a point-of-care test. However, the way to stimulate saliva need to optimise further based on the feedbacks of the participants. Also, differences between paracetamol's concentration in resting saliva and stimulated saliva were identified for the first time, which further highlighted the importance to establish and standardise collection methods for salivary analytes.

Future work will develop a point of care test format which can be readily carried out in-clinic with a portable mass spectrometer. In conclusion, PA-MS offers a simple, fast, convenient and non-invasive means to potentially risk stratify patients suspected of paracetamol overdose.

PA-MS unique features were demonstrated by comparing with two known similar methods. FIG. 12 schematically depicts a comparison of known methods of applying analytes to a substrate with the method of applying analytes to a substrate of the present invention. In one known method of applying analytes to a substrate, the analyte is applied once on the same paper. In another known method of applying analytes to a substrate, the analyte is applied once on the same paper. In the present method, the paper is already arrow-shaped even before sample application or any PC. Apply separately on different paper substrates for different analytes. That is to say, each Paper Arrow is tailored for a given chemical class or individual target. For example, if there was one single sample (say blood), then the blood sample would be dispensed onto 3 distinct PA's each one tailored for a given analyte of interest (A, B, C).

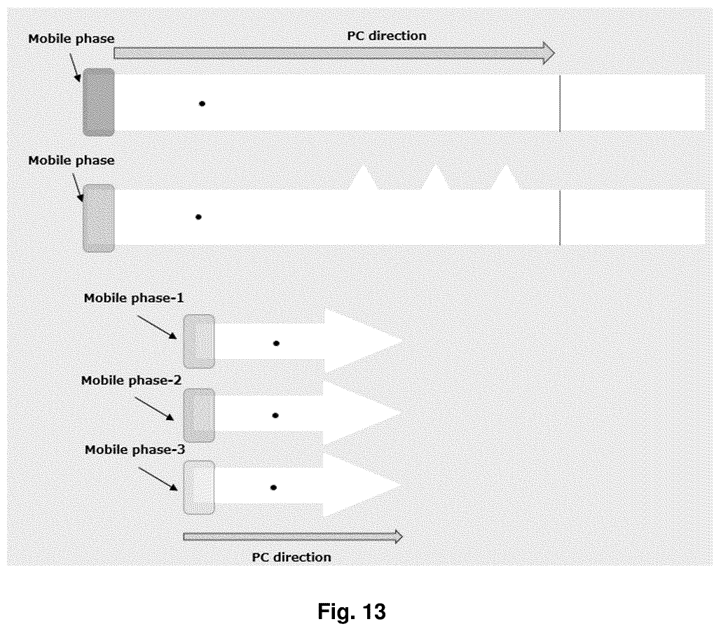

FIG. 13 schematically depicts a comparison of known methods of paper chromatography after sample application with the method of paper chromatography presented herein. In one known method PC was done with the same mobile phase, and stops before the solvent front reaches the far end of the paper strip. In another known method PC was done with the same mobile phase, and stops before the solvent front reaches the far end of the paper strip. This is standard for paper chromatography methods. In the present method, for each analyte, PC is done separately, each with specifically chosen mobile phases. And the mobile phase is allowed to reach the far end of the paper strip. The solvent front reaching the end of the already arrow-shaped paper is important. Moreover, the PC flow direction is in the same direction as the subsequent ‘electrospray’.

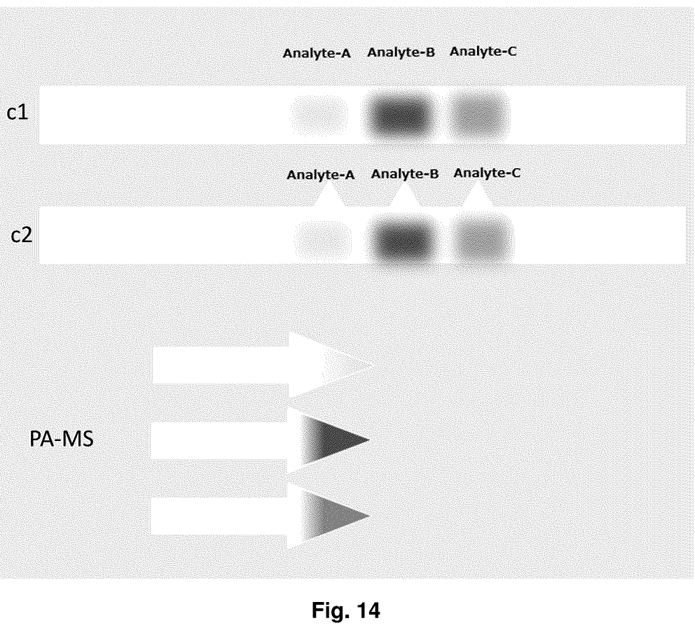

FIG. 14 schematically depicts a comparison of the results of known methods of paper chromatography after sample application with the results of the method presented herein. One known method partially separates analytes with unavoidable diffusion along PC direction. Another known method partially separate analytes with unavoidable diffusion along PC direction. Using the present method the analytes are efficiently separated/extracted by the specific mobile phases and concentrated close to the tip of the arrowhead of the arrow-shaped paper. Note: the significant issue with diffusion is completely circumvented for Paper Arrow.

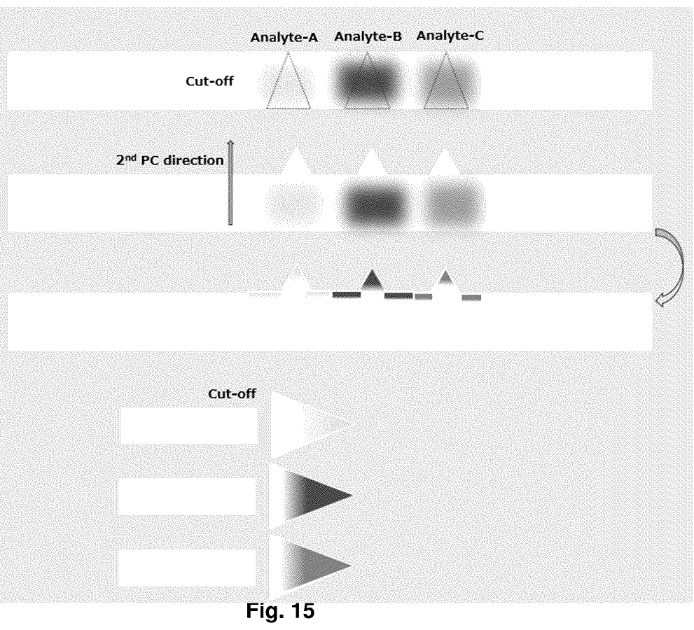

FIG. 15 schematically depicts known methods of preparing a substrate for spray with the method presented herein. One known method results in huge loss of analytes that have diffused out of the triangular area. Another known method requires 2^{nd} PC for enrichment and after 2^{nd} PC, analytes are lost too at both sides of tips. In the present method, no (or very little) analytes are lost at all. The present method outperforms UPLC-MS/MS for limit of detection.

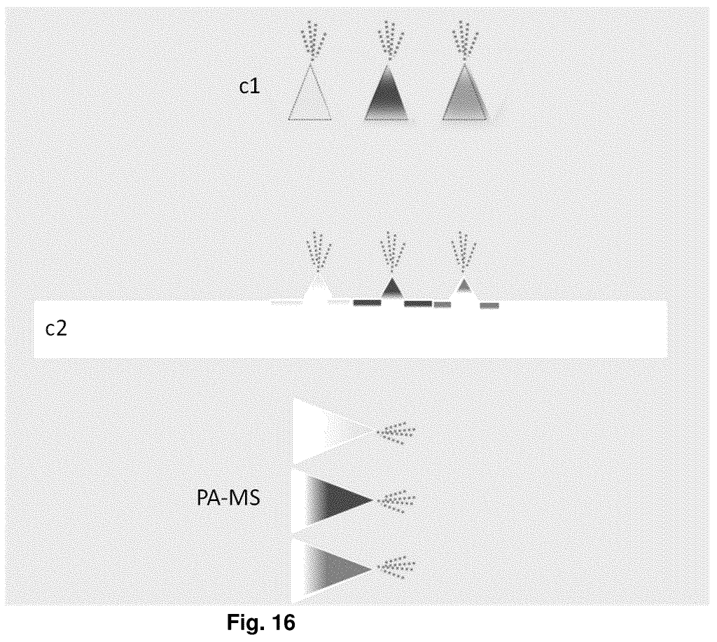

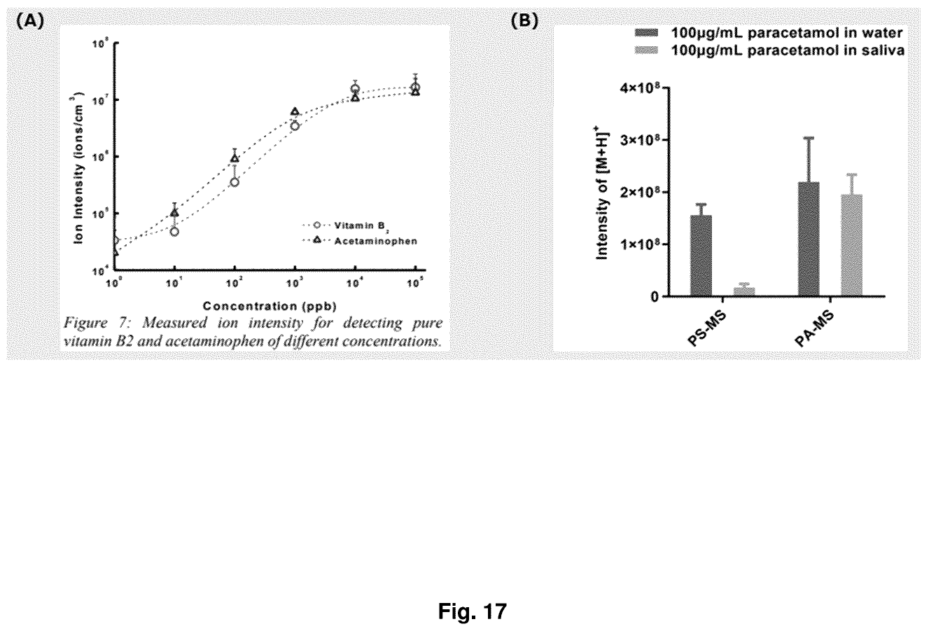

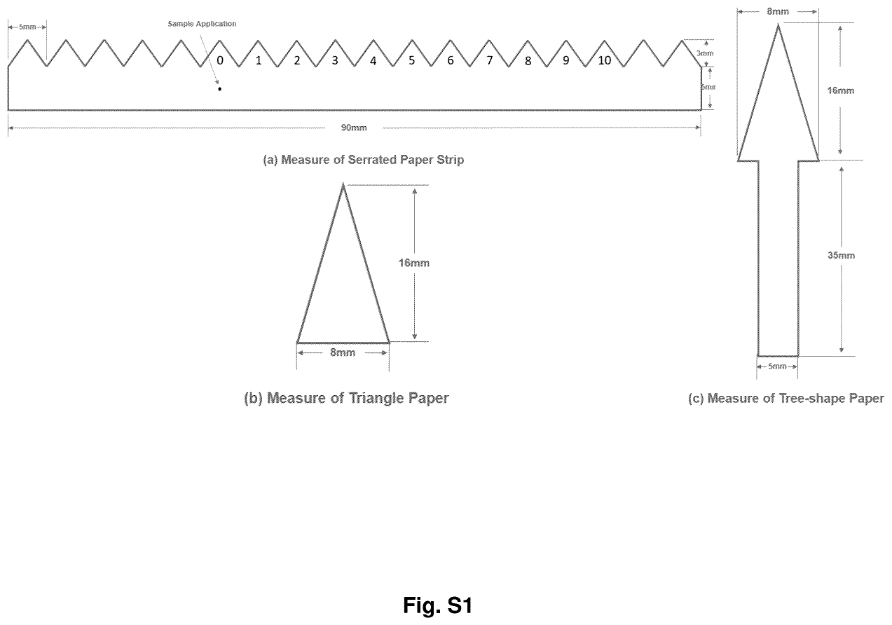

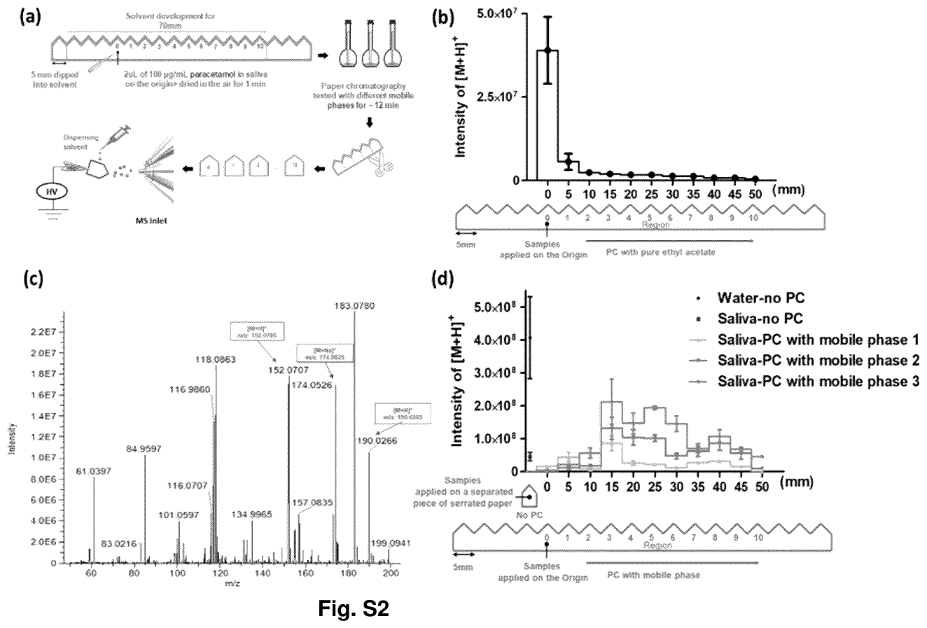

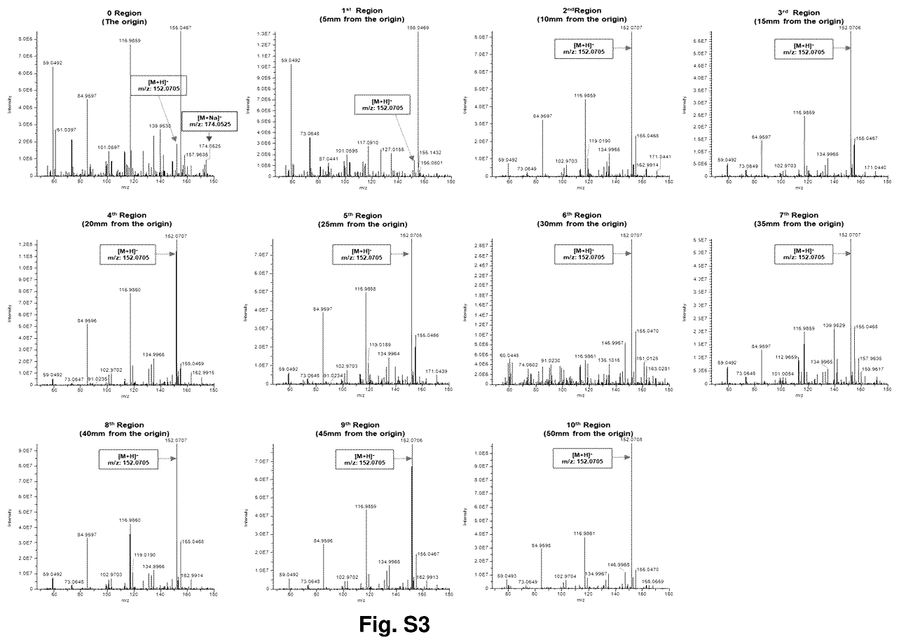

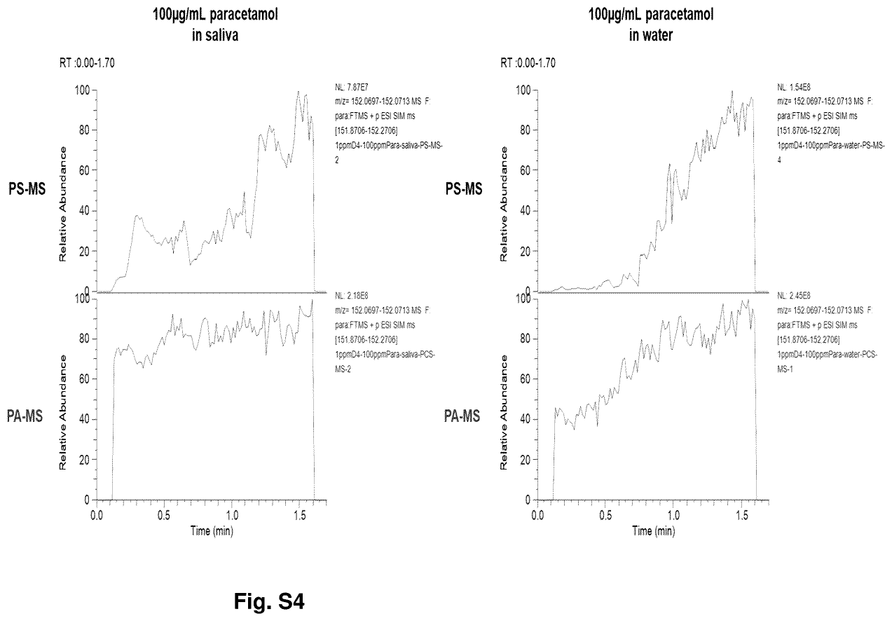

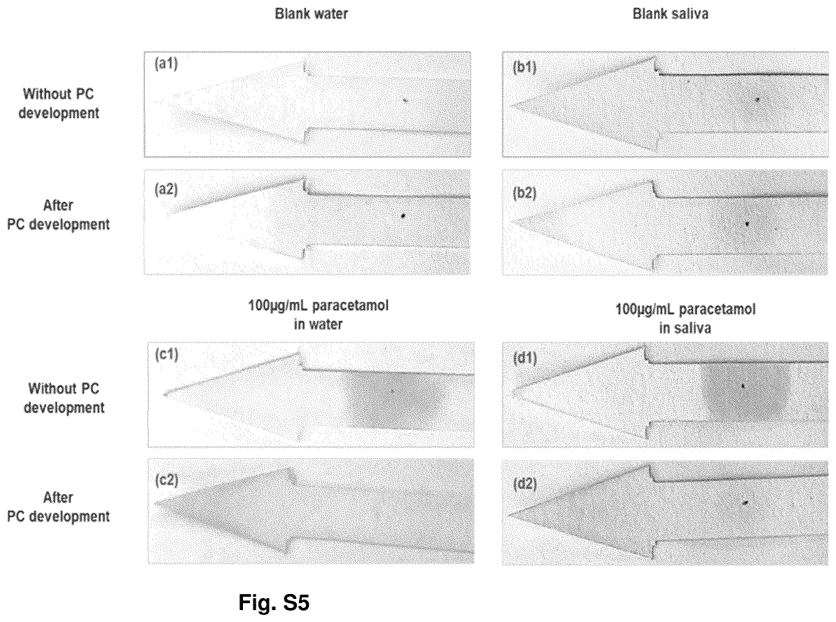

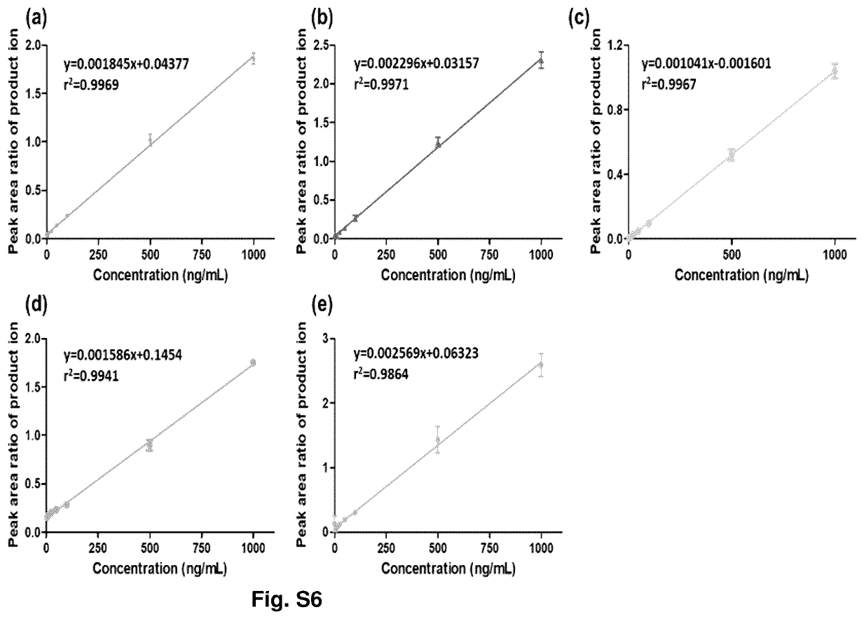

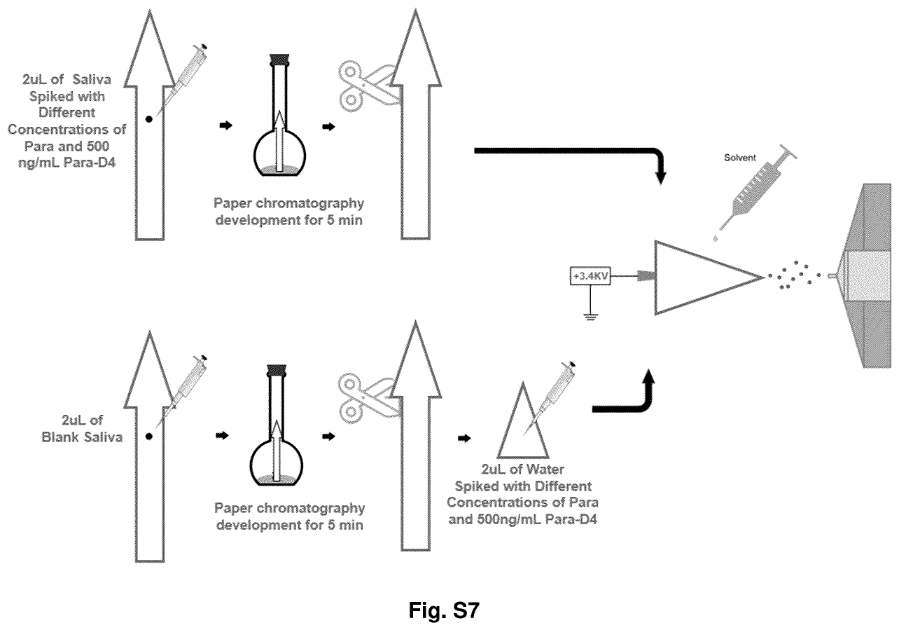

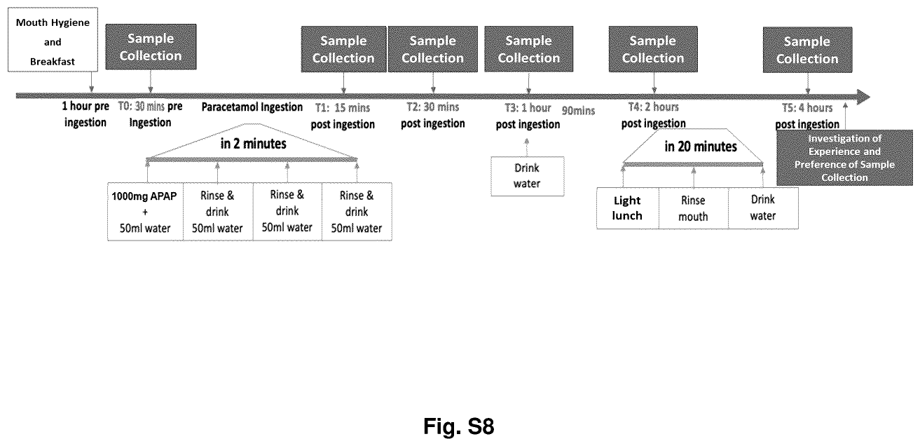

FIG. 16 schematically depicts known methods of spray after paper chromatography with the method presented herein. In one known method the spray direction is orthogonal to PC direction. In another known method, the spray direction is parallel to 2^{nd} PC direction, and orthogonal to the 1^{st} PC direction. In the present method, the spray direction is parallel to the PC direction.

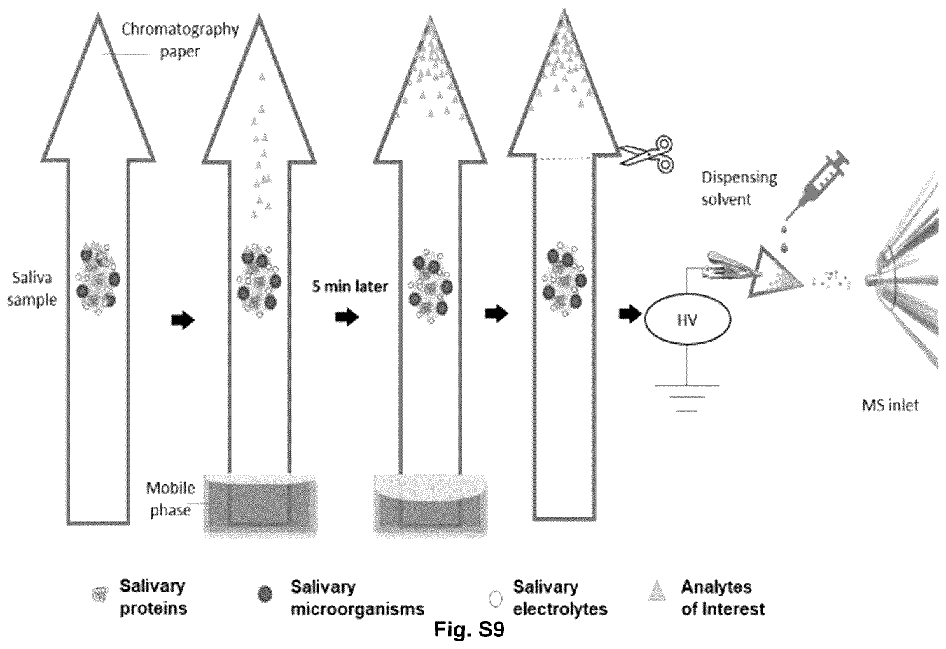

FIG. 17A is a graph of results of a known method of 2D PC. A known method detected acetaminophen (also called paracetamol) with different concentration levels by their 2D PC method. As shown in the below figure (A), 105 ppb (100 ug/mL) acetaminophen's ion intensity only reached a level of 10⁷.

FIG. 18B is a graph comparing the results of a method of PS-MS and PA-MS. The present method detected acetaminophen with PA-MS at the same concentration level. Without any loss of analytes, the signal intensity was 20 times higher than 2D-PC-method (B).[2] We also demonstrated that our method is more sensitive the UPLC-MS/MS (gold standard). This is in addition to other (obvious) benefits. e.g., time efficiency: a 1-step PC process (Paper Arrow) should be faster and less cumbersome/complex (i.e., easier to use), etc.

### Summary

Compared to other studies for the direct detection of paracetamol in biofluids, PA-MS demonstrated significantly better sensitivity. For instance, a recent study reported a LOQ of 2.9 μg/mL for paracetamol in serum samples, which had been diluted five-fold and treated with an electrokinetic extraction syringe to remove proteins.[77] In contrast, our PA-MS method allowed for the direct analysis of raw samples without the need for complex apparatus or dilution. The process of PC simultaneously achieved deproteination and desalting of the samples while concentrating the analyte of interest. These unique features of paper-arrow exhibit better sensitivity whilst making it a more practical solution, enabling analysis direct from raw saliva.

Lower LODs and LOQs for detecting paracetamol have been reported in some studies that treated biofluid samples with traditional methods like protein precipitation. For instance, R. K. Kam et al. reported a LOQ of 125 ng/ml for paracetamol in 20 μL of blood serum using LC-MS (LOQ defined as an S/N ratio higher than 10).[78] However, if using the same quantification criterion, the LOQ in this study (Paper Arrow) would be far superior, 5 ng/ml with a S/N of 135.93 (Table S2†). Other studies have used ±15% of CV and accuracy as the criteria to define the LOQ, and reported a value of 20 ng/ml for 50 μL of sample.[79] In comparison, PA-MS/MS showed that the CV and accuracy for 50 ng/mL paracetamol in saliva were 3.4% and 3.2%, respectively, with only 2 μL of saliva sample (Table S2†). Furthermore, the sample pre-treatment methods used in those studies were more complicated and time-consuming than PA-MS/MS, our method achieved comparable sensitivity with lower sample volume and a much simpler treatment procedure.

### Conclusion

PA-MS combines sample collection, extraction, separation, enrichment and ionisation onto a single piece of paper, the entire process, from sample to result, can be completed in under 10 min, while achieving analytical performance that surpasses the current state of the art.

PA-MS separates and enriches a target analyte(s) of interest to the tip of arrowhead after a 1-D paper chromatography which is performed parallel to spray direction and for which the mobile phase front reaches the end of the paper (tip of the arrowhead).

Comparing to the published methods, PA-MS is vastly superior. There is minimal loss of analyte, yielding higher sensitivity than previous methods, furthermore offering analytical performance (accuracy, precision, etc) which complies with stringent clinical analysis criteria.

### Supporting Information

### Methods

### 1. Chemicals and Reagents

Paracetamol (meets USP testing specifications, 98.0-102.0%), paracetamol-D4 (100 ug/mL in methanol), Iron (III) chloride (reagent grade, 97%), potassium ferricyanide (III) (99%), and reagent-grade formic acid (≥95%) were purchased from Sigma-Aldrich (St. Louis, Mo, USA). Ammonium formate (99%), methanol (99.8%, HPLC grade) and hydrochloric acid (1M) were purchased from Fisher Scientific (Loughborough, UK). Ethyl acetate (≥99.5%) was purchased from Merck KGaA (Darmstadt, Germany). Deionised water was purified using a Milli-Q Advantage A10 water purification system (Millipore, MA, USA) before use in this study. Chromatography paper (25 mm, Grade 1) was purchased from Whatman (Maidstone, UK).

Standard stock solutions of paracetamol (2000 μg/mL) was prepared with pure methanol and stored at −20° C. Stock solutions of Iron (III) Chloride (0.2M), potassium ferricyanide (III) (0.02M) and ammonium formate (1M) were prepared with deionised water and stored at 4° C. 100 μg/mL paracetamol diluted from 2000 μg/mL stock solution was used as working solution.

Chromatography paper (Whatman 1 CHR, size: 250*250 mm, Maidstone, UK) was cut into different shapes of paper by a laser cutter, and washed by methanol 5 min-water 5 min-methanol in ultrasonic bath.

Horse whole blood, bovine serum, human urine, and human sweat were purchased from commercial companies.

### 2. Human Whole Saliva Collection

The collection and preparation of human saliva was conducted under the ethical approval (approval number: 10058) by the ethical committee of University of Liverpool. Informed consent of participating subjects was obtained. Restricted from intake of any food or drinks for at least 1 hr., human whole saliva was directly collected in a vial after passively pooling at the bottom of the mouth for 2 min⁶⁰. Saliva samples were collected and analysed on the same day without any sample stored for re-usage.

### 3. Instruments and Software

### 3.1 Instruments and Software for PS-MS/MS and PA-MS/MS

PS-MS/MS and PA-MS/MS were performed with a Thermo Scientific Orbitrap Exploris 240 mass spectrometer (Thermo Fisher, Waltham, MA, USA). For paper spray, the paper was held by a copper clip at the rear to provide an electrical connection and placed 3 mm from the inlet of the mass spectrometer. The spray solvent, 9:1 methanol: water (v/v) with 0.5% formic acid and 10 mM ammonium formate, was automatically pumped onto the centre of paper with a speed of 500 μL/min during 0.01-0.09 min by the instrument's syringe pump. Conditions of ion source were: spray voltage, ±3.5 kV; ion transfer tube temperature, 320° C.; without nebulizer gas supply. The voltage was applied to induce an electrospray event from 0.10 min and stopped at 0.45 min. The 0.35 min voltage application cycle was repeated three more times to generate a total of four peaks in one chromatogram. The total run time was 1.66 min. The data acquisition was under the control of Xcaliber software (Thermo Finnigan, USA).

### 3.2 Instruments and Software for UPLC-MS/MS Method

UPLC-MS/MS was carried out on a Waters ACQUITY UPLC system (Waters Corporation, Milford, MA). Injection volume was 3 μL. UPLC separation was performed on a Waters ACQUITY UPLC BEH C18 column (2.1 mm×50 mm, 1.7 μm) with a BEH C18 guard column (2.1 mm×5mm, 1.7 μm). The mobile phase consisted of combinations of A (0.1% formic acid in water, v/v) and B (0.1% formic acid in methanol, v/v) at a flow rate of 0.5 mL/min with an elution gradient as follows: 0-2 min, 5% B; 2 min, 50% B; 2.5 min, 95% B. A 2.5-min post-run time was set to fully equilibrate the column. Column and sample chamber temperatures were 40° C. and 6° C. respectively. Mass spectrometry analysis was conducted by a Waters Xevo Triple mass spectrometer (Waters, Milford, USA) with electrospray ionization (ESI) in positive mode.

Nebulization and cone gases were nitrogen and set at 1000 L/h and 150 L/h, respectively. The source temperature was kept at 600° C. The source capillary voltage was 3.7 kV. Argon was applied as collision gas. Peaks were integrated using MassLynx V4.1 SCN 901 (Waters, Milford, USA) and the peak area ratio of the quantifiers of paracetamol and paracetamol-D4 were used for quantification.

### 4. Experimental Procedures

### 4.1 Development of PA-MS/MS for Salivary Paracetamol Detection

### 4.1.1 PC Mobile Phase Selection

The experiment of this section was done with serrated paper stripes (FIG. S1), which were cut by laser cutter and 0-10 was noted onto the strips as shown in FIG. S2a. These stripes were washed in the sequence of methanol (5 min)-water (5 min)-methanol(5 min) by an ultrasonic cleaner. The sample of 100 μg/mL paracetamol in saliva was made by spiking 5 μL of 2000 μg/mL stock paracetamol solution into 95 μL freshly collected saliva. Then 1 μL sample was applied onto the centre of 1^{st} region for twice with a total volume of 2 μL. The paper stripes dried up in air under room temperature in 1 min.

Pure ethyl acetate was tried first according to literatures.^{61,62} As shown in FIG. S2, 500 μL of ethyl acetate was pooled at the bottom of a flask with a depth less than 5 mm. The above paper stripe was put into the flask with one end immersed into ethyl acetate. The ethyl acetate was absorbed to the far end by capillary force of paper cellulose. When the front of mobile phase reached the 10^{th} region from the origin after 12 min, the paper strip was taken out of flask and dried in air for 1 min. Then the regions of 0-10 were cut apart manually. Each piece of paper was sprayed into Thermo Scientific Orbitrap Exploris 240 and mass spectrometry was as described the above. Full scan with m/z range of 500-200 and Selected lon Monitoring (SIM) of adducts of paracetamol were conducted to get data of interest. For SIM, the mass range was specified by typing the chemical formula of paracetamol (C₉H₈NO₂) with selected adducts of H⁺, Na⁺, or K⁺ in the Trace Type column of the Chromatogram Ranges view. The mass tolerance was chosen as 5 ppm. As seen in FIG. S2b, pure ethyl acetate failed to separate paracetamol out of saliva with abundant [M+Na]⁺ formed (FIG. S2c).

According to a published paper⁶³, formic acid and ammonium formate were added into ethyl acetate, and three kinds of combination were compared: 9:1 (v/v) of ethyl acetate: formic acid, which was abbreviated as 9:1 EA:FA in FIG. S2d; 10 mM ammonium acetate in 9:1 (v/v) of ethyl acetate: formic acid, which was noted as 10 mM NH₄HCO₂-9:1 EA:FA; and 50 mM ammonium acetate in 9:1 (v/v) of ethyl acetate:formic acid, which was 50 mM NH4HCO2-9:1 EA:FA in FIG. S2d. The process of paper chromatography (PC) with these 3 solvents was done as the same with pure ethyl acetate. However, to offer a reference for judgement of mobile phase, this time two more experiments were conducted with PS-MS. Samples of 100 μg/mL paracetamol in saliva or in water were prepared as describe above, and 2 μL sample was applied onto triangle paper (FIG. S1), which was prepared as same as the serrated paper stripes. Without PC, the pieces of triangle paper were detected with the same conditions of mass spectrometry as described above. Results showed that 50 mM ammonium acetate in 9:1 (v/v) of ethyl acetate: formic acid could produce higher intensity of [M+H]⁺ than the other two solvents (FIG. S2d).

### 4.1.2 Locating Saliva Constituents After PC

In this section, the experiment was done with serrated paper too. Differently from the above, 2 μL blank saliva was applied onto the origin and PC was done with 50 mM ammonium acetate in 9:1 (v/v) of ethyl acetate: formic acid. After 12 min process of PC as described in 4.1.1, the 0-10 regions were cut off, and 2 μL 100 μg/mL paracetamol in water was applied onto each region. After dried up in air under room temperature, full scan of 50-180 and SIM of [M+H]⁺, [M+Na]⁺, and [M+K]⁺ were conducted on Thermo Scientific Orbitrap Exploris 240. The representative mass spectra of full scan of each region are listed in FIG. S3. The process of experiment and data of SIM were shown in FIG. 2.

### 4.1.3 Ensuring Sufficient Analyte Separation

In this part, the experiment was done as: 2 μL 100 μg/mL paracetamol in saliva was applied onto serrated paper stripe and 12 min PC was conducted with mobile phase of 50 mM ammonium acetate in 9:1 (v/v) of ethyl acetate: formic acid. SIM of [M+H]⁺, [M+Na]⁺, and [M+K]⁺ were conducted by Thermo Scientific Orbitrap Exploris 240. The experiment process and data were shown in FIG. 3.

### 4.1.4 PA Substrate Design: Seamless Integration of PC and PS

The arrow-shaped paper was used in this section. The measures of arrow-shaped were shown in FIG. S1. Paper was cut by laser too. A pencil dot was marked on the stem at the position of 5 mm, 10 mm, and 15 mm from the base of triangle. And then the arrow-shaped paper washed in methanol (5 min)-water (5 min)-methanol (5 min) by an ultrasonic cleaner. Similarly, 2 μL 100 μg/mL paracetamol in saliva was applied onto the pencil dot, and 5 min of PC was done with 50 mM ammonium acetate in 9:1 (v/v) of ethyl acetate: formic acid. After dried in air, the triangle part of the arrow-shaped paper was manually cut off. [M+H]⁺, [M+Na]⁺, and [M+K]⁺ of paracetamol was detected with SIM by Thermo Scientific Orbitrap Exploris 240. Results can be found in FIG. 4.

### 4.1.5 PA-MS Verification

Experiments were done to evaluate PA-MS's effect on salivary paracetamol detection by comparing with traditional PS-MS. With regard to PS-MS, 2 μL 100 μg/mL paracetamol in saliva or in water was applied onto triangle paper and detected by PS-MS without PC; While for PA-MS, 2 μL 100 μg/mL paracetamol in saliva or in water was applied onto 10 mm position of arrow-shaped paper and detected by PSC-MS after 5 min PC with 50 mM ammonium acetate in 9:1 (v/v) of ethyl acetate: formic acid. The intensity of [M+H]⁺, [M+Na]⁺, and [M+K]⁺ was detected by SIM on Thermo Scientific Orbitrap Exploris 240. The result was compared among groups (FIG. 5 (a) and FIG. 5(b) in the manuscript). Furthermore, 7 participants were recruited and their resting saliva was collected as described in section of 2. Demographic information of 7 participants are summarised in Table S1. The 7 participants' saliva samples were spiked with the same concentration of 100 μg/mL paracetamol, and then detected by PA-MS and PS-MS. As seen in FIG. 5(c) and Table 1.

Herein, the distribution of paracetamol on arrow-shaped paper after PC was also compared with that on the traditional triangle paper. According to published colorimetry experiment of paracetamol,^{64, 65} 20 mMFeCl₃, 10 mM K₃Fe(CN)₆ and 0.2M HCl in deionised water was sprayed onto the paper by a fine mist sprayer. The spray solution was freshly made from stock solutions of Iron(III) Chloride and Potassium ferricyanide(III) no longer than 2 hours in advance of experiment. After sprayed with the dyeing solution, paracetamol on paper was dyed into the colour of Prussian blue, whose formula is KFe^{III}[Fe^{II}(CN)₆]. Pictures in FIG. 4(c) and FIG. 5(b) were taken in 2-5 min after sprayed with the dyeing solution, and the colour was slightly different because of variation of taken time. The two chemical reactions of dyeing process were:

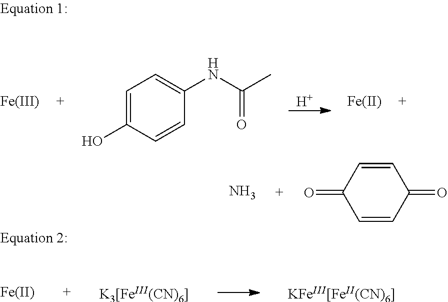

### 4.1.6 Statistical Analysis

All the above experiments were repeated at least for 3 times. The mean and Standard Deviation (SD) of average peak intensities of [M+H]⁺, [M+Na]⁺, and [M+K]⁺ was calculated, analysed and plotted by the software of GraphPad Prisma 5.0 (GraphPad Software, San Diego, CA, US). One-way ANOVA and Tukey's multiple comparison test was done for comparison among groups.

### 4.2 Validation of PA-MS/MS for Salivary Paracetamol Detection

### 4.2.1 Linearity, LOD and LOQ of Salivary Paracetamol (Para) Detection by PA-MS/MS,

To validate the method of PA-MS/MS, paracetamol-D4 (Para-D4) was used as internal standard (IS). Serial dilution was used to prepare samples. Taking samples of saliva as an example, 2.5 μL of 100 μg/mL Para-D4 was spiked with 497.5 μL human whole saliva, and then the saliva with 500 ng/ml Para-D4 was divided into 7 portions of 99 μL, 50 μL, 80 μL, 50 μL, 50 μL, 60 μL, and 50 μL into 1.5 mL Eppendorf tubes. Then 1 μL working solution of Para (100 μg/mL) was spiked into the first portion of 99 ul to make the first saliva sample of 1000 ng/ml Para with 500 ng/ml Para-D4. Half of the first sample was added into the second portion of 50 μL saliva, and the second saliva sample of 500 ng/ml Para with 500 ng/ml Para-D4 was made. 20 μL of the second sample was spiked into the third portion of 80 μL and the third saliva sample of 100 ng/ml Para with 500 ng/ml Para-D4 was prepared, and so on. Totally, 7 concentration levels of 5, 10, 25, 50, 100, 500, and 1000 ng/ml Para with 500 ng/ml Para-D4 were prepared in saliva and in water for PA-MS/MS. Similarly, 90, 360, 540, 720, 900, 1800, 4500 ng/ml Para with 5000 ng/ml Para-D4 in saliva were prepared for ultra-performance liquid chromatography (HPLC)-MS/MS.

Five sets of experiments were conducted in this section: paracetamol in raw saliva detected by PA-MS/MS, paracetamol in pure water detected by PA-MS/MS, paracetamol in raw saliva detected PS-MS/MS, paracetamol in pre-treated saliva detected PS-MS/MS, and paracetamol in pre-treated saliva by ultra-performance liquid chromatography (HPLC)-MS/MS. In the last two sets, the treatment of saliva samples was: samples were spiked with methanol (1:4, v/v), cooled for 30 min under −20° C., and then centrifuged for 20 min with 14000 rpm under 4° C. The supervant was diluted 4 times with deionised water. For PS-MS/MS and PA-MS/MS, the collision energy was set as 70%, and the Selective Reaction Monitoring (SRM) transitions were: m/z 152.0706→110.0600 (quantifier) and m/z 152.0706→65.071 (qualifier) for paracetamol, and m/z 156. 0957→114.0850 (quantifier) and m/z 156.0957→69.090 (qualifier) for paracetamol-D4. For UPLC-MS/MS, the multiple reaction monitoring (MRM) transitions monitored were: m/z 151.94→109.95 as a quantifier and m/z 151.94→92.84 as a qualifier for paracetamol and m/z 155.96→114.05 as quantifier and m/z 155.96→96.69 as qualifier for paracetamol-D4. The optimized parameters for the four MRM transitions were: cone voltage, 46, 46, 38 and 38V; collision energy: 16, 22, 15 and 21 eV, respectively. The dwell time was 0.1 s per transition. The average peak area ratios of quantifier of Para over that of Para-D4 were plotted to the theoretical concentrations. The linear regression equations and the squares of correlation coefficients (r²) were noted in FIG. 6. The limits of detection (LODs) and the limits of quantitation (LOQs) of the three sets of experiments were listed in Table 2. More results were described in Table S1-S6.

4.2.2 Linearity, Accuracy and Precision of PA-MS/MS Within the Range of 0.2-200 μg/mL

In this section, the performance of PA-MS/MS in a higher range of 0.2-200 μg/MI was evaluated, which covers the targeted population's concentrations of 15-100 μg/mL.[10] Serial dilution was used to prepare saliva samples of 0.2, 1, 5, 25, 50, 100, 200 μg/mL Para with 1 μg/mL Para-D4. Then, 2 μL of samples was applied onto arrow-shaped paper, developed for 5 min of PC, and then detected on Thermo Scientific Orbitrap Exploris 240 mass spectrometer. The calibration curve of 0.2-200 g/mL was constructed in FIG. 6. The accuracy and precision of Data was seen in Table S7.

### 4.2.3 Extraction Recovery and Matrix Factors of PA-MS/MS

The experiment to evaluate the extraction recovery of salivary detection by PA-MS/MS was conducted as shown in FIG. S6. First, samples of 5, 15, 500, and 1000 ng/ml Para with 500 ng/ml Para-D4 were prepared in saliva and in water. For pre-chrom spiked set, 2 μL of saliva samples spiked with Para and Para-D4 was applied onto arrow-shaped paper to do PA-MS/MS as described in 4.2.1. For the post-chrom spiked set, 2 μL of blank saliva was applied onto the stem of arrow-shaped paper. After the triangle was cut off, 2 μL of water spiked with Para and Para-D4 was applied onto the triangle paper. Then mass spectrometry was conducted on Thermo Scientific Orbitrap Exploris 240. Peak area ratios of product ion of 110.06 over 114.08were used to calculate the extraction recovery at 4 concentration levels as in Equation 3.⁶⁶

The matrix factors of PA-MS/MS and PS-MS/MS were evaluated at 3 concentration levels of 100, 500 and 1000 ng/ml Para with 500 ng/ml Para-D4 in saliva and water. For the experiments of PS-MS/MS, 2 μL of saliva or water samples spiked with Para and Para-D4 was applied onto triangle paper to do PS-MS/MS as described in 4.2.1. With regard to PA-MS/MS, the samples were applied onto arrow-shaped paper and detected after 5 min PC. The matrix factors were calculated with equations of 4-6 according to the guideline of EMA.⁶⁷

$$Extraction Recovery = \frac{Peak Area Ratio of pre - chrom spiked saliva}{Peak Area Ratio of post - chrom spiked saliva} \times 100 \% \qquad Equation 3$$

$$Matrix factor of Para = \frac{Peak Area of Production of Para in saliva}{Peak Area of Production of Para in water} \times 100 \% \qquad Equation 4$$

$$Matrix factor of Para - D 4 = \frac{Peak Area of Production of Para - D 4 in saliva}{Peak Area of Production of Para - D 4 in water} \times 100 \% \qquad Equation 5$$

$$IS normalised Matrix factor = \frac{Matrix factor of Para}{Matrix factor of Para - D 4} \times 100 \% \qquad Equation 6$$

### 4.2.3 Accuracy and Precision of PA-MS/MS

The method accuracy and precision were evaluated for PA-MS/MS with the same concentration levels on three days and replicated for 3 times on each day. As described before, 2 μL of saliva samples was applied onto arrow-shaped paper, developed for 5 min of PC, and then detected on Thermo Scientific Orbitrap Exploris 240 mass spectrometer. Data was listed in Table S8, which were all fulfilled the requirements of FDA and EMA.⁶⁸

### 4.3 Exploration in Other Biofluids

The above methodology was tried in horse whole blood, bovine serum, human urine, and human sweat too. Paracetamol's concentration was 100 μg/mL, and the procedure was the same as saliva. The intensity of [M+H]⁺ of paracetamol in positive mode of SIM on Thermo Scientific Orbitrap Exploris 240.

TABLE S1
Demographic information of 7 participants

| | | Age | | Resting saliva flow |
|---|---|---|---|---|
| Code | Gender | (years) | Ethnics/Nationality | rate (g/min) |
| A | Female | 44 | Chinese | 0.38 |
| B | Female | 57 | White British | 0.21 |
| C | Female | 34 | Korean | 0.24 |
| D | Female | 27 | Thai | 0.75 |
| E | Male | 34 | Turkish | 0.12 |
| F | Male | 22 | White British | 0.16 |
| G | Male | 36 | White British | 0.12 |

TABLE S2
Precision and Accuracy of Paracetamol in Raw
Saliva Detected by PA-MS/MS(5-1000 ng/mL)

| Theoretical | | | | Calculated | | Mean of |
|---|---|---|---|---|---|---|
| Concentration | Peak Area Ratio | | | Concentration | | Ratio of |
| (ng/mL) | Mean | SD | CV | (ng/mL) | Accuracy | S/N^{[a]} |
| 5 | 0.032 | 0.005 | 14.2% | −6.38 | −227.6% | 135.93 |
| 10 | 0.061 | 0.002 | 3.5% | 9.07 | −9.3% | 82.022 |
| 25 | 0.078 | 0.003 | 4.0% | 18.28 | −26.9% | 210.40 |
| 50 | 0.139 | 0.005 | 3.4% | 51.62 | 3.2% | 185.70 |
| 100 | 0.240 | 0.008 | 3.5% | 106.36 | 6.4% | 307.36 |
| 500 | 1.021 | 0.063 | 6.1% | 529.84 | 6.0% | 425.71 |
| 1000 | 1.860 | 0.055 | 3.0% | 984.23 | −1.6% | 2.64*E16 |

^{[a]}Ratio of S/N is the signal of product ion of m/z 110.06 over the signal of background noise.

TABLE S3
Precision and Accuracy of Paracetamol in Raw
Saliva Detected by PS-MS/MS(5-1000 ng/mL)

| Theoretical | | | | Calculated | |
|---|---|---|---|---|---|
| Concentration | Peak Area Ratio | | | Concentration | |
| (ng/mL) | Mean | SD | CV | (ng/mL) | Accuracy |
| 5 | 0.050 | 0.012 | 24.0% | −5.15 | −203.0% |
| 10 | 0.063 | 0.011 | 16.9% | −0.22 | −102.2% |
| 25 | 0.114 | 0.010 | 8.9% | 19.83 | −20.7% |
| 50 | 0.195 | 0.037 | 19.1% | 51.10 | 2.2% |
| 100 | 0.304 | 0.016 | 5.4% | 93.66 | −6.3% |
| 500 | 1.436 | 0.201 | 14.0% | 534.17 | 6.8% |
| 1000 | 2.591 | 0.177 | 6.8% | 983.82 | −1.6% |

TABLE S4
Precision and Accuracy of Paracetamol in Pure
Water Detected by PA-MS/MS(5-1000 ng/mL)

| Theoretical | | | | Calculated | |
|---|---|---|---|---|---|
| Concentration | Peak Area Ratio | | | Concentration | |
| (ng/mL) | Mean | SD | CV | (ng/mL) | Accuracy |
| 5 | 0.037 | 0.003 | 9.4% | 2.36 | −52.7% |
| 10 | 0.041 | 0.006 | 14.0% | 3.96 | −60.4% |
| 25 | 0.081 | 0.007 | 8.7% | 21.64 | −13.4% |
| 50 | 0.138 | 0.009 | 6.1% | 46.50 | −7.0% |
| 100 | 0.267 | 0.041 | 15.4% | 102.54 | 2.5% |
| 500 | 1.246 | 0.066 | 5.3% | 528.72 | 5.7% |
| 1000 | 2.306 | 0.106 | 4.6% | 990.61 | −0.9% |

TABLE S5
Precision and Accuracy of Paracetamol in Pre-treated
Saliva Detection by UPLC-MS/MS (5-1000 ng/mL)

| Theoretical | | | | Calculated | |
|---|---|---|---|---|---|
| Concentration | Peak Area Ratio | | | Concentration | |
| (ng/mL) | Mean | SD | CV | (ng/mL) | Accuracy |
| 5 | 0.005 | 0.0006 | 11.8% | 6.68 | 33.5% |
| 10 | 0.011 | 0.0010 | 9.4% | 11.72 | 17.2% |
| 25 | 0.026 | 0.0046 | 17.7% | 26.62 | 6.5% |
| 50 | 0.049 | 0.0053 | 10.8% | 48.47 | −3.1% |
| 100 | 0.096 | 0.0044 | 4.6% | 93.75 | −6.2% |
| 500 | 0.519 | 0.0501 | 9.7% | 500.57 | 0.1% |
| 1000 | 1.040 | 0.0633 | 6.1% | 1000.10 | 0.0% |

TABLE S6
Precision and Accuracy of Paracetamol in Pre-treated
Saliva Detection by PS-MS/MS (5-1000 ng/mL)

| Theoretical | | | | Calculated | |
|---|---|---|---|---|---|
| Concentration | Peak Area Ratio | | | Concentration | |
| (ng/mL) | Mean | SD | CV | (ng/mL) | Accuracy |
| 5 | 0.156 | 0.0442 | 28.3% | 6.82 | 36.4% |
| 10 | 0.172 | 0.0308 | 17.9% | 16.77 | 67.7% |
| 25 | 0.203 | 0.0276 | 13.6% | 36.30 | 45.2% |
| 50 | 0.233 | 0.0188 | 8.1% | 55.20 | 10.4% |
| 100 | 0.282 | 0.0210 | 7.5% | 86.23 | −13.8% |
| 500 | 0.900 | 0.0964 | 10.7% | 476.06 | −4.8% |
| 1000 | 1.752 | 0.0333 | 1.9% | 1012.84 | 1.3% |

TABLE S7
Precision and Accuracy of Salivary Paracetamol
Detection by PA-MS(0.2-200 μg/mL)

| Theoretical | | | | Calculated | |
|---|---|---|---|---|---|
| Concentration | Peak Area Ratio | | | Concentration | Accu- |
| (μg/mL) | Mean | SD | CV | (μg/mL) | racy |
| 0.2 | 0.86125 | 0.012842 | 1.5% | 0.249924737 | 25.0% |
| 1 | 1.6325 | 0.015155 | 0.9% | 1.023883593 | 2.4% |
| 5 | 5.5385 | 0.138163 | 2.5% | 4.943602609 | −1.1% |
| 25 | 24.399 | 0.295092 | 1.2% | 23.87034621 | −4.5% |
| 100 | 102.0683 | 1.022391 | 1.0% | 101.812477 | 1.8% |
| 200 | 199.1457 | 10.13514 | 5.1% | 199.2307744 | −0.4% |

TABLE S8
Intra-Day and Inter-Day Precision of Paracetamol Detection in Saliva by PA-MS.

| Theoretical | | Intra-Day Peak | | | Inter-Day Peak | | |
|---|---|---|---|---|---|---|---|
| Concentration | | Area Ratio | | CV of | Area Ratio | | CV of |
| (μg/mL) | Sets | Mean | SD | Intra-Day | Mean | SD | Inter-Day |
| 0.2 | Day 1 | 0.31 | 0.02 | 7.4% | 0.29 | 0.02 | 7.7% |
| | Day 2 | 0.28 | 0.02 | 8.7% | | | |
| | Day 3 | 0.26 | 0.04 | 14.2% | | | |
| 1 | Day 1 | 1.10 | 0.03 | 2.5% | 1.09 | 0.03 | 2.5% |
| | Day 2 | 1.05 | 0.08 | 7.7% | | | |
| | Day 3 | 1.10 | 0.00 | 0.2% | | | |
| 25 | Day 1 | 25.20 | 0.84 | 3.3% | 25.38 | 0.32 | 1.3% |
| | Day 2 | 25.19 | 1.00 | 4.0% | | | |
| | Day 3 | 25.75 | 0.70 | 2.7% | | | |
| 100 | Day 1 | 101.40 | 1.02 | 1.01% | 94.98 | 5.62 | 5.9% |
| | Day 2 | 92.60 | 6.20 | 6.7% | | | |
| | Day 3 | 90.94 | 3.89 | 4.30% | | | |

### 5 Method of Clinical Validation

### 5.1 Participants' Recruitment and Demographics

In this study, a total of 20 participants were initially recruited. However, 2 of them were later excluded due to abnormal liver or kidney function, and an additional participant was excluded as it was not possible to obtain blood samples after trying 11 times. Consequently, a total of 17 participants were included in the final analysis. Among these participants, there were 5 males and 12 females, with 12 being of white ethnicity and 5 being non-white. The average age of the participants was 36.8 years, with ages ranging from 19 years to 55 years.

### 5.2 Samples and Information Collection

Baseline samples of blood, resting saliva, and stimulated saliva were collected, as mentioned previously. Additionally, liver function tests, urea, and creatinine levels were measured to ensure the participants' health status before proceeding with the study. These measurements are shown in Table S9.

TABLE S9
Body composition of participants (N = 17)

| Parameters | Mean ± SD | Range |
|---|---|---|
| Height(cm) | 166.6 ± 8.0 | (156.0, 182.0) |
| Weight(kg) | 75.2 ± 15.9 | (44.3, 111.4) |
| Water(kg) | 36.3 ± 7.5 | (26.2, 54.4) |
| Protein(kg) | 9.8 ± 2.0 | (7.0, 14.8) |
| Minerals(kg) | 3.5 ± 0.7 | (2.63, 5.12) |
| Muscle mass(kg) | 27.5 ± 6.2 | (19.2, 42.7) |
| Fat mass(kg) | 25.7 ± 10.7 | (6.9, 44.3) |
| Percent of body fat (PBF) (%) | 33.2 ± 10.4 | (12.5, 46.4) |
| Body mass index (BMI) (kg/m2) | 27.0 ± 5.1 | (18.0, 35.6) |
| Body surface area (BSA) (m2) a | 1.9 ± 0.2 | (1.39, 2.34) |

$a : BSA ( m 2 ) = \sqrt{\frac{Height ( cm ) \times Weight ( kg )}{3600}}$

During the waiting period, an InBody 570 Body Composition Analyzer (BioSpace, Seoul, Korea) was employed to measure participants' body composition. This device utilises bioelectrical impedance analysis (BIA) to calculate body composition based on five variables: electric resistance, height, weight, age, and sex. BIA is a non-invasive and commonly used method for assessing body composition, providing valuable information about body fat percentage, muscle mass, and other related parameters (Table S10).

TABLE S10
Urea, creatinine and the parameters of liver function (N = 17)

| Parameters | N | Mean ± SD | Range |
|---|---|---|---|
| Urea (mmol/L) | 17 | 4.6 ± 2.3 | (2.7, 12.7) |
| Creatinine | 17 | 67.4 ± 13.0 | (45.0, 101.0) |
| (μmol/L) | | | |
| Bilirubin (μmol/L) | 15a | 12.6 ± 9.1 | (6.0, 42.0) |
| AST (iu/L) | 17 | 22.3 ± 14.0 | (13.0, 72.0) |
| ALT (iu/L) | 17 | 23.1 ± 11.9 | (11.0, 50.0) |
| ALP (iu/L) | 17 | 64.2 ± 18.2 | (42.0, 117.0) |
| Total protein (g/L) | 17 | 71.9 ± 4.5 | (66.0, 82.0) |
| Albumin (g/L) | 17 | 44.5 ± 2.9 | (40.0, 49.0) |

aBilirubin in 2 out of 17 participants was lower than the LOQ of 5 μmol/L.

Once participants' liver function was confirmed to be within normal ranges, 2 tablets of 500 mg paracetamol were administered. Subsequently, samples were collected five times over a 4-hour period (see FIG. S8).

After completing the sample collection, participants were asked to complete a self-designed questionnaire. This questionnaire utilised a 5-Likert scale to assess participants' perceptions and preferences regarding the three sample collection procedures. Furthermore, open-ended questions were included to gather qualitative experiences and comments from the participants. This feedback will provide valuable insights into their overall experience with the study's sampling methods and may help improve future research protocols.

### 5.3 Gold Standard Test Procedure

To proceed with the gold standard test procedure, a 1.3 mL blood sample was collected in a Li-Heparin tube and then centrifuged for 5 minutes. The resulting supernatant plasma was analysed using an automated clinical acetaminophen assay (Abbott Laboratories, IL, USA). The enzymatic reagents reacted with paracetamol, and the final reaction product, 4-(4-iminophenol)-2,5-dimethylcyclohexadiene-1-one, was measured at 660nm using Alinity ci-series (Abbott Laboratories, IL, USA). The current gold standard test demonstrates the following analytical performance characteristics: Limit of Detection (LOD) of 1 mg/L, Limit of Quantification (LOQ) of 3 mg/L, inter-assay CV of 0.6-4.6%, and a concentration range of 3-337 mg/L.

### 5.4 PA-MS Procedure

The novel method to be cross-validated is named Paper-Arrow Mass Spectrometry (PA-MS). This method requires only 2 μL of raw sample and approximately 5 minutes for paper chromatography (PC) separation without any sample pre-treatment prior to MS analysis (see FIG. S9).

The absorbent substrate used is arrow-shaped chromatography paper with pre-holed isotopic internal standard Paracetamol-D4. The PA-MS procedure involves adding a 2 μL sample onto the shaft of the arrow-shaped paper, which is then dried in 1 minute. Next, the flat end of the shaft is dipped into a solvent mixture consisting of 50 mM NH4HCO2 in 9:1 ethyl acetate: formic acid (v/v). This solvent mixture carries paracetamol and separates it from the matrix, concentrating it at the arrowhead. Finally, the analyte is physically isolated by cutting the arrowhead from the arrow for direct MS analysis. With a Thermo Scientific Orbitrap Exploris 240 mass spectrometer (Thermo Fisher, Waltham, MA, USA). Briefly, the arrowhead paper was held by a copper clip at the rear to provide an electrical connection and placed 5 mm from the inlet of the mass spectrometer. The spray solvent, 9:1 methanol:water (v/v) with 0.5% formic acid and 10 mM ammonium formate, was automatically pumped onto the centre of the paper at a rate of 500 μL/min during 0.01-0.09 min using the instrument's syringe pump. The ion source conditions were set as: spray voltage, +3.5 kV; ion transfer tube temperature, 320° C.; without nebuliser gas supply. Nitrogen was used as the collision gas. Multiple reaction monitoring transitions were: m/z 152.0706→110.0600 (quantifier) and m/z 152.0706→65.071 (qualifier) for paracetamol, and m/z 156.0957→114.0850 (quantifier) and m/z 156.0957→69.090 (qualifier) for paracetamol-D4. The voltage was applied to induce an electrospray event for 1.66 min. The data acquisition was under the control of Thermo Scientific Xcalibur software and data processing was completed using the Xcalibur Quan Browser.

The entire process is simple and can be completed within approximately 10 minutes or less, providing superior analytical performance with samples of plasma, resting saliva, and stimulated saliva (refer to Table S11).

TABLE S11
LODs and LOQs of paracetamol detection in plasma,
resting saliva and stimulated saliva by PA-MS

| | LODa | LOQb | Concentration | Inter-assay |
|---|---|---|---|---|
| Matrix | (mg/L) | (mg/L) | Range (mg/L) | CV % |
| Plasma | 0.07 | 0.21 | 0.2-200 | 1.1-6.0 |
| Resting saliva | 0.06 | 0.18 | 0.2-200 | 0.7-4.2 |
| Stimulated saliva | 0.04 | 0.13 | 0.2-200 | 0.7-3.5 |

aLOD = 3.3*SD of Response/Slope.
bLOQ = 10*SD of Response/Slope.

### 5.5 Statistical Analysis

The four sets of detection were described using the mean and standard deviation (SD). To validate the performance of the novel tests, correlation, agreement, and bias estimation tests were conducted following the guidelines from the European Medicines Agency (EMA), 69 and U.S. Food and Drug Administration (FDA). 70 The correlation between the novel test and the gold standard test was assessed using Lin's concordance correlation coefficient (CCC). 71 This coefficient quantifies the agreement between the two measures of the same variable. CCC is preferred over Reming regression as it considers both the accuracy and precision of the two tests. Additionally, a Bland-Altman difference plot was generated after regression analysis to assess the agreement between the two methods and estimate any bias. Furthermore, ratios of PA-MS over the gold standard were plotted along time curves to analyse trends over time. The criteria used for these tests were summarized in Table S12.

The results of participants' perceptions and preferences regarding the three sample collection procedures were described by mean rank, and the comparisons between them were done by Kruskal-Wallis test.

Statistical analysis was performed using the following software: IBM SPSS Statistics, version 25 (IBM Corp.), GraphPad Prism 5.0 (GraphPad Software, San Diego, CA, US), and Microsoft Excel. Statistical significance was set at p values<0.05.

TABLE S12
Summary of criteria of cross-validation methods

| Statistical analysis | Parameters | Criteria |
|---|---|---|
| Lin's Concordance | ρc (r) | >0.85 |
| Correlation | | |
| Bland-Altman plot | 95% CI of mean of differences | Including 0 |
| | 95% limit of agreements | Within ±6 mg/L |
| | Correlation between differences | No correlation exists |
| | and means | |
| Time curve of ratios of PA- | Ratios of two methods' results | 67% ratios are within |
| MS / Gold standard test | | 1.0 ± 0.2 |
| | The means of ratios at all time | Within 1.0 ± 0.2 |
| | points | |
| | One-way ANOVA between 5 time | p value > 0.05 |
| | points | |

### REFERENCES

1. F. P. M. Jjunju, D. E. Damon, D. Romero-Perez, I. S. Young, R. J. Ward, A. Marshall, S. Maher and A. K. Badu-Tawiah, Sci. Rep., 2020, 10, 10698.
2. M. Jurisch and R. Augusti, Anal. Methods, 2016, 8, 4543-4546.
3. S. Maher, F. P. M. Jjunju, D. E. Damon, H. Gorton, Y. S. Maher, S. U. Syed, R. M. A. Heeren, I. S. Young, S. Taylor and A. K. Badu-Tawiah, Sci. Rep., 2016, 6, 35643.
4. F. P. M. Jjunju, S. Maher, D. E. Damon, R. M. Barrett, S. U. Syed, R. M. A. Heeren, S. Taylor and A. K. Badu-Tawiah, Anal. Chem., 2016, 88, 1391-1400.
5. B. S. Frey, D. R. Heiss and A. K. Badu-Tawiah, Anal. Chem., 2022, 94, 4417-4425.
6. D. E. Damon, M. Yin, D. M. Allen, Y. S. Maher, C. J. Tanny, S. Oyola-Reynoso, B. L. Smith, S. Maher, M. M. Thuo and A. K. Badu-Tawiah, Anal. Chem., 2018, 90, 9353-9358.
7. B. S. Frey, D. E. Damon, D. M. Allen, J. Baker, S. Asamoah and A. K. Badu-Tawiah, Analyst, 2021, 146, 6780-6787.
8. N. E. Manicke, P. Abu-Rabie, N. Spooner, Z. Ouyang and R. G. Cooks, Journal of the American Society for Mass Spectrometry, 2011, 22, 1501-1507.
9. R.-Z. Shi, E. T. M. El Gierari, N. E. Manicke and J. D. Faix, Clin. Chim. Acta, 2015, 441, 99-104.
10. R. D. Espy, N. E. Manicke, Z. Ouyang and R. G. Cooks, Analyst, 2012, 137, 2344-2349.
11. T.-T. Sham, A. K. Badu-Tawiah, S. J. McWilliam and S. Maher, Sci. Rep., 2022, 12, 14308.
12. K. E. Yannell, K. R. Kesely, H. D. Chien, C. B. Kissinger and R. G. Cooks, Anal. Bioanal. Chem., 2017, 409, 121-131.
13. R. D. Espy, S. F. Teunissen, N. E. Manicke, Y. Ren, Z. Ouyang, A. van Asten and R. G. Cooks, Anal. Chem., 2014, 86, 7712-7718.
14. D. E. Damon, K. M. Davis, C. R. Moreira, P. Capone, R. Cruttenden and A. K. Badu-Tawiah, Anal. Chem., 2016, 88, 1878-1884.
15. B. L. Smith, C. Boisdon, D. Romero-Perez, T.-T. Sham, B. Bastani, Y. Zhou, S. McWilliam, A. K. Badu-Tawiah and S. Maher, Int. J. Mass spectrom., 2022, 471, 116737.
16. S. Maher, F. P. M. Jjunju and S. Taylor, Rev. Mod. Phys., 2015, 87, 113-135.
17. D. E. Damon, Y. S. Maher, M. Yin, F. P. M. Jjunju, I. S. Young, S. Taylor, S. Maher and A. K. Badu-Tawiah, Analyst, 2016, 141, 3866-3873.
18. S. Suraritdechachai, C. Charoenpakdee, I. Young, S. Maher, T. Vilaivan and T. Praneenararat, J. Agric. Food. Chem., 2019, 67, 3055-3061.
19. H. Lee, C.-S. Jhang, J.-T. Liu and C.-H. Lin, J. Sep. Sci., 2012, 35, 2822-2825.
20. Y.-N. Yao, D. Di, Z.-C. Yuan, L. Wu and B. Hu, Anal. Chem., 2020, 92, 6207-6212.
21. Q. Wang, Y. Zheng, X. Zhang, X. Han, T. Wang and Z. Zhang, Analyst, 2015, 140, 8048-8056.
22. W. Zhang, X. Wang, Y. Xia and Z. Ouyang, Theranostics, 2017, 7, 2968-2981.
23. C. Vega, C. Spence, C. Zhang, B. J. Bills and N. E. Manicke, J. Am. Soc. Mass. Spectrom., 2016, 27, 726-734.
24. C. Zhang and N. E. Manicke, Anal. Chem., 2015, 87, 6212-6219.
25. N. M. Sarih, D. Romero-Perez, B. Bastani, M. Rauytanapanit, C. Boisdon, T. Praneenararat, H. A. Tajuddin, Z. Abdullah, A. K. Badu-Tawiah and S. Maher, Sci. Rep., 2020, 10, 21504.
26. D. E. Damon, Y. S. Maher, D. M. Allen, J. Baker, B. S. Chang, S. Maher, M. M. Thuo and A. K. Badu-Tawiah, Langmuir, 2019, 35, 13853-13859.
27. P. Panuwet, R. E. Hunter, Jr., P. E. D′Souza, X. Chen, S. A. Radford, J. R. Cohen, M. E. Marder, K. Kartavenka, P. B. Ryan and D. B. Barr, Crit Rev Anal Chem, 2016, 46, 93-105.
28. Y. Zhou, T. T. Sham, C. Boisdon, B. L. Smith, J. C. Blair, D. B. Hawcutt and S. Maher, Analyst, 2023, DOI: 10.1039/d3an00850a.
29. A. S. Chidiac, N. A. Buckley, F. Noghrehchi and R. Cairns, Expert Opinion on Drug Metabolism & Toxicology, 2023, 19, 297-317.
30. H. H. Jeong, K. Cha, K. H. Choi and B. H. So, BMC pharmacology & toxicology, 2022, 23, 5.
31. China Pat., CN 214011152 U, 2020.
32. M.-H. Cheng and C.-H. Lin, Journal, 2018, DOI: 10.1109/MEMSYS.2018.8346786, 1233-1236.
33. C. H. Chen and C. H. Lin, 2017.
34. N. Jehmlich, K. H. Dinh, M. Gesell-Salazar, E. Hammer, L. Steil, V. M. Dhople, C. Schurmann, B. Holtfreter, T. Kocher and U. Völker, J Periodontal Res, 2013, 48, 392-403.
35. E. M. A. Committee for Medicinal Products for Human Use, ICH guideline M10 on bioanalytical 5), method validation and study sample analysis (Step https://www.ema europa.eu/en/ich-m10-bioanalytical-method-validation-scientific-guideline#current version-section).
36. D. J. Jollow, S. S. Thorgeirsson, W. Z. Potter, M. Hashimoto and J. R. Mitchell, Pharmacology, 1974, 12, 251-271.
37. R. K. Kam, M. H. Chan, H. T. Wong, A. Ghose, A. M. Dondorp, K. Plewes and J. Tarning, Future Sci OA, 2018, 4, Fso331.
38. A. Trettin, A. A. Zoerner, A. Böhmer, F.-M. Gutzki, D. O. Stichtenoth, J. Jordan and D. Tsikas, Journal of Chromatography B, 2011, 879, 2274-2280.
39. K. Usui, H. Kobayashi, Y. Fujita, E. Kubota, T. Hanazawa, T. Yoshizawa, Y. Kamijo and M. Funayama, J Food Drug Anal, 2019, 27, 786-792.
40. H. Ishii, T. Obara, K. Kusano and I. Kijima-Suda, Drug Test Anal, 2020, 12, 1196-1202.
41. H. Ishii, T. Obara and I. Kijima-Suda, Drug Testing and Analysis, 2020, 12, 929-937.
42. L. D. Betowski, W. A. Korfmacher, J. O. Lay, Jr., D. W. Potter and J. A. Hinson, Biomed Environ Mass Spectrom, 1987, 14, 705-709.
43. U. Heintze, D. Birkhed and H. Björn, Swed Dent J, 1983, 7, 227-238.
44. H. Hiraba, M. Yamaoka, M. Fukano, T. Fujiwara and K. Ueda, Somatosens Mot Res, 2008, 25, 222-229.
45. F. Mizuhashi and K. Koide, J Adv Prosthodont, 2020, 12, 204-209.
46. M. Macdonald, N. A. Ghani, Y. Wan, J. Cooper-White, G. Dimeski and C. Punyadeera, Bioanalysis, 2014, 6, 697-704.
47. K. Kato, M. Hillsgrove, L. Weinhold, D. A. Gorelick, W. D. Darwin and E. J. Cone, J Anal Toxicol, 1993, 17, 338-341.
48. T. Barranco, J. J. Ceron, P. López-Jornet, J. Pastor, J. M. Carrillo, M. Rubio, P. L. Tornel, R. Cugat, F. Tecles and A. Tvarijonaviciute, Journal, 2018, 619.
49. N. Idkaidek and T. Arafat, Molecular Pharmaceutics, 2012, 9, 2358-2363.
50. N. Idkaidek and T. Arafat, Drug Res (Stuttg), 2014, 64, 559-562.
51. G. B. Proctor, Periodontology 2000, 2016, 70, 11-25.
52. B. C. Muddugangadhar, R. Sangur, I. V. Rudraprasad, D. B. Nandeeshwar and B. H. Kumar, J Indian Prosthodont Soc, 2015, 15, 356-366.
53. J. P. Glynn and W. Bastain, J Pharm Pharmacol, 1973, 25, 420-421.
54. Journal, 2011.
55. S. S. Al-Obaidy, A. Li Wan Po, P. J. McKiernan, J. F. T. Glasgow and J. Millership, Journal of Pharmaceutical and Biomedical Analysis, 1995, 13, 1033-1039.
56. P. M. M. Bezerra, R. C. Costa, I. L. A. Ribeiro, P. R. F. Bonan, S. A. d. Sousa and A. M. G. Valença, Pesquisa Brasileira em Odontopediatria e Clínica Integrada, 2019, 19, 1-11.
57. J. H. Soderstrom, D. M. Fatovich, C. Mandelt, S. Vasikaran, D. L. McCoubrie, F. F. Daly and S. A. Burrows, Br J Clin Pharmacol, 2012, 74, 154-160.
58. S. Chandran, A. Alsadi and E. Ahmad, Exploring User Centered Design in Healthcare: A Literature Review, 2020.
59. R. Gorodischer, P. Burtin, P. Hwang, M. Levine and G. Koren, Therapeutic Drug Monitoring, 1994, 16, 437-443.
60. G.-V. Sonia, S.-S. Aurea, M.-C. José María, A.-S. José Manuel and M. Alex, PLoS ONE, 2018, 13, e0198021-e0198021.
61. M. H. Abdallah, A. M. Hanan, S. S. Ahmed and S. I. Hany, Journal, 2020.
62. D. Mohamed, O. Hassan, N. Bahnasawy, A. S. Elnoby and S. Mowaka, Microchemical Journal, 2019, 150, 104146.
63. Journal, 2020.
64. P. Nagendra, E-Journal of Chemistry, 2011, 8, 149-152.
65. L. Sundar and F. Rowell, Analyst, 2014, 139, 633-642.
66. J. W. M. Roland and V. Stephan, Journal of Applied Bioanalysis, 2018, 4, 67-73.
67. K. Boesen, P. C. Gotzsche and J. P. A. loannidis, EPIDEMIOLOGY AND PSYCHIATRIC SCIENCES, 2021, 30, e35.
68. M. Kaza, M. Karaźniewicz-Łada, K. Kosicka, A. Siemiątkowska and P. J. Rudzki, Journal of Pharmaceutical and Biomedical Analysis, 2019, 165, 381-385.
69. E. M. Agency, Journal, 2011.
70. U. D. o. H. a. H. S. Food and Drug Administration, Journal, 2018.
71. H. Akoglu, Turk J Emerg Med, 2018, 18, 91-93.

### Notes

Although a preferred embodiment has been shown and described, it will be appreciated by those skilled in the art that various changes and modifications might be made without departing from the scope of the invention, as defined in the appended claims and as described above.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims and drawings), and/or all of the steps of any method or process so disclosed, may be combined in any combination, except combinations where at most some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims and drawings), or to any novel one, or any novel combination, of the steps of any method or process so disclosed.

1. A method comprising:
providing a sample comprising analytes, for example in a matrix, on a substrate, for example a monolithic substrate, comprising a stationary phase;
separating the analytes, for example mutually and/or relative to the matrix, on the substrate using a mobile phase; and
analysing the separated analytes using mass spectrometry, wherein the substrate provides, at least in part, an ion source for ionising the separated analytes.

2. The method according to any previous claim, wherein the mass spectrometry comprises and/or is: paper spray mass spectrometry; leaf spray mass spectrometry; coated blade spray mass spectrometry; and/or solid-substrate electrospray; optionally, wherein the mass spectrometry comprises using a solvent, for example a spray solvent; and/or wherein the separating the analytes on the substrate using the mobile phase comprises and/or is: paper chromatography; and/or solid phase extraction.

3. The method according to any previous claim, wherein analysing the separated analytes using mass spectrometry comprises applying an electric potential to the substrate, thereby providing, at least in part, the ion source for ionising the separated analytes.

4. The method according to any previous claim, wherein the substrate comprises a protrusion, for example having an apex, thereby providing, at least in part, the ion source for ionising the separated analytes; optionally, wherein the substrate comprises a plurality of protrusions, for example each having an apex, thereby providing, at least in part, a plurality of ion sources for ionising the separated analytes.

5. The method according to any previous claim, wherein the substrate comprises and/or is: a porous substrate; a non-porous substrate; a layered substrate; and/or a fibrous substrate, for example a cellulosic substrate such as paper.

6. The method according to any previous claim, wherein the substrate has a first end and a mutually-opposed second end, defining an axis therebetween, wherein separating the analytes on the substrate using the mobile phase comprises separating the analytes axially.

7. The method according to claim 6, wherein separating the analytes on the substrate using the mobile phase comprises contacting the substrate with the mobile phase proximal and/or at the first end of the substrate.

8. The method according to any of claims 6 to 7, wherein the second end of the substrate provides, at least in part, the ion source for ionising the separated analytes.

9. The method according to any previous claim, comprising parting the substrate, for example into a first part and a second part, after separating the analytes, for example mutually and/or relative to the matrix, on the substrate using the mobile phase, for example wherein the second part provides, at least in part, the ion source for ionising the separated analytes.

10. The method according to any previous claim, wherein the analytes comprise: an active pharmaceutical ingredient and/or a metabolite thereof, for example paracetamol; a xenobiotic; a biomolecule; a toxic compound; an explosive.

11. The method according to any previous claim, wherein the sample comprises a biological sample, such as saliva, urine, whole blood and/or serum; an environmental sample, such as water, leachate.

12. A method of Paper Arrow Mass Spectrometry (PA-MS) comprising:
paper-chromatography (PC) and paper-spray mass-spectrometry (PS-MS) of a sample comprising analytes, for example using the same paper for the PC and for the PS-MS.

13. An apparatus combining paper-chromatography (PC) and paper-spray mass-spectrometry (PS-MS).

14. A method of selecting a mobile phase, the method comprising:
providing a sample comprising a matrix, on a substrate, for example a monolithic substrate, comprising a stationary phase;
separating the matrix on the substrate in a direction using a first mobile phase;
dividing the substrate transverse, preferably orthogonally, to the direction;
providing analytes on the substrate, for example on each division of the divided substrate after dividing and/or before dividing; and
analysing the analytes using mass spectrometry, wherein the substrate provides, at least in part, an ion source for ionising the analytes, for example wherein each division of the divided substrate provides, at least in part, a respective ion source for ionising the analytes.

15. A method of identifying a separation distance between analytes and a matrix, the method comprising:
providing a sample comprising the analytes and the matrix, on a substrate, for example a monolithic substrate, comprising a stationary phase;
mutually separating the analytes and the matrix, on the substrate in a direction using a mobile phase;
dividing the substrate transverse, preferably orthogonally, to the direction; and
analysing the separated analytes using mass spectrometry, wherein the substrate provides, at least in part, an ion source for ionising the separated analytes, for example wherein each division of the divided substrate provides, at least in part, a respective ion source for ionising the separated analytes.
